(19) United States
(12) Patent Application Publication
(10) Pub. No.: US 2012/0149878 A1
(43) Pub. Date: Jun. 14, 2012

(54) PROTEIN PURIFICATION

(76) Inventors: Ronald GILLESPIE, Seattle, WA (US); Suresh Vunnum, Bellevue, WA (US); Thao Nguyen, Renton, WA (US); Sean Macneil, Seattle, WA (US)

(21) Appl. No.: 13/303,918

(22) Filed: Nov. 23, 2011

Related U.S. Application Data
(60) Provisional application No. 61/421,158, filed on Dec. 8, 2010.

(51) Int. Cl. C07K 1/18

(52) U.S. Cl. 530/387.3; 530/416; 530/388.1

(57) ABSTRACT

Methods of reducing high molecular weight species (HMW) formation in a sample containing a protein purified using ion exchange (IEX) chromatography are disclosed, as are a number of related methods, e.g., methods of reducing on-column denaturation of a protein in a protein sample purified using an ion exchange (IEX) column or resin. The methods share characteristics of including arginine, glycine and/or histidine in the buffers used during the ion exchange (IEX) chromatography.

### CROSS-REFERENCE TO RELATED APPLICATIONS

This application claims the benefit of U.S. Provisional Application No. 61/421,158, filed Dec. 8, 2010, which is hereby incorporated by reference.

### FIELD OF THE INVENTION

The present invention relates to improvements in IEX chromatography, useful in the production of therapeutic biological molecules.

### BACKGROUND

Therapeutic proteins, or biologicals, e.g., monoclonal antibodies (mAbs) and Fc fusion proteins, occupy a large share of the current protein therapeutic market with many more potential biologicals, e.g., mAbs, in the development pipeline (Walsh, G. (2004), Biopharm. Intnl. 17, 18). The ability to quickly move a candidate biologic to the clinic and; ultimately, to the market is essential for the success of biopharmaceutical companies. To achieve these goals, the biotechnology industry has adopted a platform approach for the manufacturing of biologics such as monoclonal antibodies (Shukla, A. A., et al., (2007). Journal of Chromatography B 848, 28-39.). Ion exchange chromatography (IEX), particularly cation exchange chromatography (CEX), is widely used in platform mAb purification processes as a polishing step due to its high capacity, selectivity for impurities, scalability, and robustness (Shukla, et al., (2007) supra; Zeid, J., et al., (2008). Biotechnology and Bioengineering 102, 971-976). CEX is an effective step for removing protein high molecular weight (HMW) species, as well as host cell protein, DNA, and residual protein A (Zeid, et al., (2008) supra; Yigzaw, Y., et al., (2009), Current Pharmaceutical Biotechnology 10, 421-426; Gagnon, P., Purification tools for monoclonal antibodies. 1996: Validated Biosystems, Inc.; Stein, A., and Kiesewetter, A. (2007). Journal of Chromatography B 848, 151-158; Staby, A., et al., (2006), Journal of Chromatography 1118, 168-179). Generally, CEX is operated in bind-and-elute mode (BEM) where the protein is bound to the resin under low conductivity conditions at a pH that is below the pI of the target molecule. Elution of the bound protein is then typically achieved by increasing the conductivity and/or inducing a pH shift. This can be performed either over a linear gradient or a step elution to predetermined conditions. Impurities, particularly HMW species, often bind more tightly than the mAb product and can be separated from the main desired fraction by adjusting the elution conditions and pool collection criteria (Yigzaw, Y., et al., (2009) supra; Gagnon, P., et al., (1996) supra; Pabst, T. M., et al., (2009) Journal of Chromatography 1216, 7950-7956).

With the adoption of platform technologies for monoclonal antibody purification, defined based on significant historical experience, it is generally expected that most molecules fit within the predefined operating space with little or no difficulty. However, despite similar tertiary structure, different mAbs can behave differently based on differences in their primary sequence and can have varying degrees of physical and chemical stability (Wang, W., et al., (2006), Journal of Pharmaceutical Sciences 96, 1-26.). Downstream platform processes should be designed to accommodate differences between mAbs; however, in some cases such streamlined platform processes prove inadequate to achieve targeted product quality attributes.

Of the various modes of chromatography used in mAb downstream processes, ion exchange chromatography is typically regarded as a mild operation with respect to potential impact on protein stability and/or integrity. However, challenges can arise even with this common unit operation. Unexpected peak shapes on CEX chromatography have been reported by Voitl and Morbidelli, where high purity human serum albumin eluted as two distinct peaks from Fractogel EMD SE Hicap (Voitl, A., Butte, A., and Morbidelli, M. (2010). Journal of Chromatography 1217, 5484-5291; Voitl, A., Butte, A., and Morbidelli, M. (2010). Journal of Chromatography 1217, 5492-5500). The two peaks in that case were attributed to two different binding conformations of human serum albumin on the CEX resin. The first peak corresponded to an instantaneous binding orientation which then could transition to the second orientation based on the CEX operating conditions. There was no increase in HMW species in the second peak.

Unexpected elution profiles have also been reported with reversed-phase (RP) and hydrophobic interaction chromatography (HIC) which has been tied to denaturation on the chromatographic surfaces. Conformational changes when binding to reversed-phase surfaces has been well established (McNay, J. L. and Fernandez, E, J. (1999), Journal of Chromatography 849, 135-148). Lu et al. showed two peaks during elution of ribonuclease A from a RP column, in which the first peak was identified as the properly folded native state while the second peak was unfolded protein (Lu, X. M., et al., (1986), Journal of Chromatography 359, 19-29). Although HIC is generally thought to be less detrimental to protein structure compared to RP, cases of peak splitting and protein unfolding have been reported. For example, Jungbauer, et al., showed that model, proteins eluted off of HIC resins in two peaks and assumed that the first peak was native protein and the second peak contained protein with a partially unfolded conformation. This assumption was made based on the rationale that unfolded protein exposes more hydrophobic surface area to the resin and will therefore be retained more than native protein. It was also noted that the degree of unfolding correlated to the binding salt concentration and the resin hydrophobicity (Jungbauer, A., et al., (2005), Journal of Chromatography 1079, 221-228). Fernandez and colleagues used hydrogen deuterium exchange to demonstrate conformational changes upon binding to HIC media and to explain the generation of two peaks from pure material (Tibbs Jones, T., and Fernandez, E. J. (2003), Journal of Colloid and Interface Science 259, 27-35). During these studies it was demonstrated that the less, retained peak had deuterium uptake similar to the native protein in the absence of resin, while the more retained peak had higher deuterium uptake and thus a higher degree of solvent exposure. They also went on to model unfolding as a function of salt concentration and temperature (Xiao, Y., et al., (2007), Journal of Chromatography 1157, 197-206) as well as to demonstrate that higher mass loads can lessen the degree of unfolding on HIC resins (Fogle, J. L., et al., (2006), Journal of Chromatography 1121, 209-218).

There are few studies demonstrating surface induced denaturation with ion exchange media. The possibility of structural perturbations during IEX has, been hinted at by both Gagnon and Fernandez but no details, were provided (Gagnon, P., (1996) supra; Fogle, J. L., and Fernandez, E. J. (2006), LCGC North America 24, 158-168). Lewis and Nail showed increased IgG aggregation during low pH treatment following anion exchange (AEX) chromatography. This was tied to the IEX column collection criteria and the susceptibility of different IgG subclasses to HMW generation at low pH (Lewis, J. D., and Nail, S. L. (1997), Process Biochemistry 32, 279-283). Hunter and Carta described two peaks, when eluting bovine serum albumin (BSA) from an AEX column, however this was attributed to the presence of BSA dimers in the feed rather than protein unfolding (Hunter, A. K., and Carta, G. (2001). Journal of Chromatography 937, 13-19).

### SUMMARY OF THE INVENTION

In one aspect, the invention includes a method of reducing high molecular weight species (HMW) formation in a sample containing a protein purified using ion exchange (IEX) chromatography. The method includes loading the protein, in a loading buffer containing at least 1 mM, 5 mM, 10 mM, 20 mM, 30 mM, 40 mM, 50 mM, 60 mM, 70 mM, 80 mM, 90 mM or 100 mM of one or more amino acids selected from the group consisting of arginine, glycine and histidine, onto an IEX resin, and eluting the protein off the IEX resin using an elution buffer containing at least 1 mM, 5 mM, 10 mM, 20 mM, 30 mM, 40 mM, 50 mM, 60 mM, 70 mM, 80 mM, 90 mM or 100 mM of one or more amino acids selected from the group consisting of arginine, glycine and histidine. In this method, the presence of one or more amino acids selected from the group consisting of arginine, glycine and histidine in the loading and elution buffers reduces HMW formation in the sample as compared with a sample of a protein purified using, IEX chromatography with loading and elution buffers that do not contain the above-recited amino acids at the above-recited concentrations.

In another aspect, the invention includes method of reducing on-column or on-resin denaturation of a protein in a protein sample purified using an ion exchange (IEX) column or resin. The method includes loading the protein, in a loading buffer containing at least 1 mM, 5 mM, 10 mM, 20 mM, 30 mM, 40 mM, 50 mM, 60 mM, 70 mM, 80 mM, 90 mM or 100 mM of one or more amino acids selected from the group consisting of arginine, glycine and histidine, on the IEX column or resin, and eluting the protein off the IEX column or resin using an elution buffer containing at least 1 mM, 5 mM, 10 mM, 20 mM, 30 mM, 40 mM, 50 mM, 60 mM, 70 mM, 80 mM, 90 mM or 100 mM of one or more amino acids selected from the group consisting of arginine, glycine and histidine. In this method, the presence of the one or more amino acids selected from the group consisting of arginine, glycine and histidine in the loading and elution buffers reduces denaturation of the protein on the IEX column or resin as compared with a protein purified on an IEX column or resin using loading and elution buffers that do not contain one or more of the above-recited amino acids at the above-recited concentrations.

In certain embodiments, the above methods may further comprise washing or equilibriating the column or resin or matrix with a wash or equilibriation buffer between the loading and eluting steps, where the wash or equilibriation buffer contains at least 1 mM, 5 mM, 10 mM, 20 mM, 30 mM, 40 mM, 50 mM, 60 mM, 70 mM, 80 mM, 90 mM or 100 mM of one or more amino acids selected from the group consisting of arginine, glycine and histidine.

In certain embodiments, each of buffers mentioned above contain at least 10 mM, 20 mM, 30 mM, 40 mM, 50 mM, 60 mM, 70 mM, 80 mM, 90 mM or 100 mM of arginine, and/or glycine. In other embodiments, each of the buffers contains at least 100 mM, 200 mM, 300 mM, 400 mM or 500 mM glycine. In other embodiments, each of buffers mentioned above contains at least 10 mM, 20 mM, 30 mM, 40 mM, 50 mM, 50 mM, 70 mM, 80 mM, 90 mM or 100 mM arginine.

In certain embodiments, the IEX column or resin is an anion exchange (AEX) column or resin, e.g., Q Sepharose Fast Flow, DEAE Sepharose Fast Flow, ANX Sepharose 4 Fast Flow, Q Sepharose XL, Q Sepharose big beads, DEAE Sephadex A-25, DEAE Sephadex A-50, QAE Sephadex A-25, QAE Sephadex A-50, Q sepharose high performance, Q sepharose XL, Sourse 15Q, Sourse 30Q, Resourse Q, Capto Q, Capto DEAE, Mono Q, Toyopearl Super Q, Toyopearl DEAE, Toyopearl QAE, Toyopearl Q, Toyopearl GigaCap Q, TSKgel SuperQ, TSKgel DEAE, Fractogel EMD TMAE, Fractogel EMD TMAE HiCap, Fractogel EMD DEAE, Fractogel EMD DMAE, Macroprep High Q, Macro-prep-DEAE, Unosphere Q, Nuvia Q, POROS HQ, POROS PI, DEAE Ceramic HyperD, or Q Ceramic HyperD.

In certain embodiments, the IEX column or resin is a cation exchange (CEX) column or resin, e.g., SP Sepharose, CM Sepharose, Toyopearl SP 650M, and Fractogel SO₃⁻, Fractogel SO3⁻ SE HiCap (M), Fractogel COO⁻ (M), YMC-BioPro S75, Capto S, SP Sepharose XL/FF, CM Sepahrose FF, SP/CM Toyopearl 650m, Toyopearl SP 550c, Toyopearl GigaCap, UNOsphere S, Eshmuno S, Macroprep High S, or POROS HS 50.

In certain embodiments, each of the buffers has a pH of between 4.0 and 6.5. In other embodiments, each of the buffers has a pH of between 6.5 and 9.0. Exemplary buffers include, e.g., acetate buffer, MES buffer, citrate buffer and bis tris buffer. In certain embodiments, the method is carried out at a temperature of between 1° C. and 10° C. or between 2° C. and 8° C., e.g., at about 4° C. In other embodiments, the method is carried out at a temperature of between 8° C. and 15° C. In other embodiments, the method is carried out at a temperature of between 15° C. and 25° C., or between about 18° C. and 22° C. In certain embodiments, the column or resin residence time is between 1 minute and 24 hours, between 1 minute and 12 hours, between 1 minute and 8 hours or between 1 minute and 4 hours.

In certain embodiments, the protein is a recombinantly-produced protein or polypeptide, e.g., a peptibody, a domain-based protein, or an antibody, e.g., and a monoclonal antibody or antigen-binding fragment thereof. In certain embodiments, the protein is a therapeutic monoclonal antibody (mAb), e.g. an IgG1 mAb, an IgG2 mAb or an IgG4 mAb. In a more specific embodiment, the mAb is an aglycosylated mAb, e.g., an aglycosylated IgG1 mAb.

In another aspect, the invention includes a method of purifying a protein or polypeptide. The method includes purifying the protein using ion exchange (“IEX”) chromatography, e.g., anion exchange (“AEX”) or cation exchange (“CEX”) chromatography, wherein the IEX chromatography employs loading and elution buffers, and the loading and elution buffers are formulated to include or comprise one or more amino acid(s) selected from the group consisting of arginine, glycine and histidine.

In another aspect, the invention includes a method of purifying a protein or polypeptide. The method comprises loading the protein or polypeptide (suspended in a loading buffer) on an ion exchange (e.g., cation exchange or anion exchange) column or resin, optionally washing or equilibrating the column or resin with a wash buffer, and eluting the protein of polypeptide using an elution buffer, wherein the loading, elution and wash (if a wash step is included) buffers are formulated to include or comprise one of more amino acid(s) selected from the group consisting of arginine, glycine and histidine.

In another aspect, the invention includes a method of reducing HMW formation in a sample of a desired protein or polypeptide. The method comprises loading the protein or polypeptide (suspended in a loading buffer) on an ion exchange (e.g., cation exchange or anion exchange) column or resin, optionally washing or equilibrating the column or resin with a wash buffer, and eluting the protein of polypeptide using an elution buffer, wherein the loading, elution and wash (if a wash step is included) buffers are formulated to include or comprise one or more amino acid(s) selected from the group consisting of arginine, glycine and histidine, and wherein the eluted protein or polypeptide exhibits significantly decreased. HMW formation relative to the loaded protein or polypeptide.

In another aspect, the invention includes a method of reducing the “percent peak B” of a desired protein or polypeptide in a sample. The method comprises loading the protein or polypeptide (suspended in a loading buffer) on an ion exchange (e.g., cation exchange or anion exchange) column or resin, optionally washing or equilibrating the column or resin with a wash buffer, and eluting the protein of polypeptide using an elution buffer, wherein the loading, elution and wash (if a wash step is included) buffers are formulated to include or comprise one or more amino acid(s) selected from the group consisting of arginine, glycine and histidine, and wherein the eluted protein or polypeptide exhibits significantly decreased percent peak B relative to the loaded protein or polypeptide.

In another aspect, the invention includes a method of isolating a desired protein or polypeptide from other components in a liquid solution. The method comprises contacting a liquid solution, comprising the desired protein or polypeptide together with other components, with an ion exchange chromatography matrix in the presence of one or more introduced amino acid(s) selected from the group consisting of arginine, glycine and histidine, allowing the ion exchange chromatography matrix to equilibrate with the solution for a time period of between about 1 minute and about 24 hours, and obtaining the protein or polypeptide in an elution solution formulated to contain one or more amino acid(s) selected from the group consisting of arginine, glycine and histidine. In one embodiment, the time period is between about 1 minute and about 4 hours. In another embodiment, the time period is between about 5 minutes and about 2 hours.

In another aspect, the invention includes a method of isolating a protein or polypeptide from a liquid solution comprising the protein or polypeptide and at least one contaminant. The method comprises loading the liquid solution onto, an ion exchange chromatography matrix in the presence of one or more amino acid(s) selected from the group consisting of arginine, glycine and histidine; optionally washing the ion exchange chromatography matrix with a wash buffer containing or comprising one or more amino acid(s) selected from the group consisting of arginine, glycine and histidine; and eluting the protein or polypeptide from the ion exchange chromatography matrix in the presence of one or more amino acid(s) selected from the group consisting of arginine, glycine and histidine, wherein the solution comprising the eluted protein or polypeptide has a substantially lower level of the contaminant relative to the solution that was loaded on the ion exchange chromatography matrix.

In another aspect, the invention includes a method of isolating a protein or polypeptide from a liquid solution comprising the protein or polypeptide and at least one contaminant. The method comprises loading the liquid solution onto an ion exchange chromatography matrix, wherein the solution contains or comprises one or more amino acid(s) selected from the group consisting of arginine, glycine and histidine; optionally washing the ion exchange chromatography matrix with a wash buffer containing or comprising one or more amino acid(s) selected from the group consisting of arginine, glycine and histidine; and eluting the protein or polypeptide from the ion exchange chromatography matrix, with an elution buffer containing or comprising one or more amino acid(s) selected from the group consisting of arginine, glycine and histidine.

In another aspect, the invention includes a method of purifying a protein or polypeptide from a liquid solution comprising the protein or polypeptide and at least one contaminant. The method comprises binding the protein or polypeptide to an ion exchange chromatography material using a loading buffer containing or comprising one or more amino acid(s) selected from the group consisting of arginine, glycine and histidine; optionally washing the ion exchange chromatography matrix with a wash buffer containing or comprising one or more amino acid(s) selected from the group consisting of arginine, glycine and histidine; and eluting the protein or polypeptide from the ion exchange chromatography matrix with an elution buffer containing or comprising one or more amino acid(s) selected from the group consisting of arginine, glycine and histidine.

In another aspect, the invention includes a method of reducing column-induced denaturation of a protein or polypeptide on a chromatography column or resin. The method includes purifying the protein using IEX chromatography, wherein the IEX chromatography employs loading and elution buffers (and optionally awash buffer), and the loading, elution and wash (if employed) buffers include glycine, arginine or histidine.

In another aspect, the invention includes a method of reducing aggregation of a purified protein or polypeptide. The method includes purifying the protein using IEX chromatography, wherein the IEX employs loading and elution buffers (and optionally a wash buffer), and the loading, elution and wash (if employed) buffers include glycine, arginine or histidine.

In another aspect, the invention includes, in a method of purifying a recombinantly-produced protein or polypeptide employing IEX chromatography having loading and elution phases, the improvement comprising including glycine, arginine or histidine in the buffers used in both the loading and elution phases of the IEX chromatography.

In another aspect, the invention includes a purified protein or polypeptide produced by any of the above methods.

In another aspect, the invention includes a purified protein or polypeptide purified at least in part using IEX chromatography, wherein the IEX chromatography comprises loading and elution phases, and employs loading and elution buffers; and wherein the loading and elution buffers both contain glycine, arginine or histidine.

The following are examples of certain specific embodiments that are contemplated in connection with any of the above-described aspects of the invention.

In one embodiment, the amino acid is selected from the group consisting of glycine and arginine.

In one embodiment, the amino acid is glycine. In one embodiment where the loading and elution buffers include glycine, the glycine concentration is greater than about 1 mM, 2 mM, 3 mM, 4 mM, 5 mM, 6 mM, 7 mM, 8 mM, 9 mM or 10 mM. In another embodiment where the loading and elution buffers include glycine, the glycine concentration in the elution buffer is equal to or greater than about 15 mM, 20 mM, 25 mM, 30 mM, 35 mM, 40 mM, 45 mM, 50 mM, 75 mM, 100 mM, 150 mM, 200 mM, 250 mM, 300 mM, 350 mM, 400 mM, 450 mM, 500 mM, 550 mM, 600 mM, 650 mM or 700 mM. In another embodiment where the loading and elution buffers include glycine, the glycine concentration in the loading and elution buffers is between about 10 mM and about 500 mM. In another embodiment where the loading and elution buffers include glycine, the glycine concentration in the loading and elution buffers is between about 50 mM and about 500 mM. In a related embodiment where the loading and elution buffers include glycine, the glycine concentration in the loading and elution buffers is between about 100 mM and about 500 mM.

In one embodiment, the amino acid is glycine. In one embodiment where the loading, wash and elution buffers include glycine, the glycine concentration is greater than about 1 mM, 2 mM, 3 mM, 4 mM, 5 mM, 6 mM, 7 mM, 8 mM, 9 mM or 10 mM. In another embodiment where the loading, wash and elution buffers include, glycine, the glycine concentration in the elution buffer is equal to or greater than about 15 mM, 20 mM, 25 mM, 30 mM, 35 mM, 40 mM, 45 mM, 50 mM, 75 mM, 100 mM, 150 mM, 200 mM, 250 mM, 300 mM, 350 mM, 400 mM, 450 mM, 500 mM, 550 mM, 600 mM, 650 mM or 700 mM. In another embodiment where the loading, wash and elution buffers include glycine, the glycine concentration in the loading, wash and elution buffers is between about 10 mM and about 500 mM. In another embodiment where the loading, wash and elution buffers include glycine, the glycine concentration in the loading, wash and elution buffers is between about 50 mM and about 500 mM. In a related embodiment where the loading, wash and elution buffers include glycine, the glycine concentration in the loading, wash and elution buffers is between about 100 mM and about 500 mM.

In another embodiment, the amino acid is arginine. In one embodiment where the loading and elution buffers include arginine, the arginine concentration is greater than about 1 mM, 2 mM, 3 mM, 4 mM, 5 mM, 6 mM, 7 mM, 8 mM, 9 mM or 10 mM. In another embodiment where the loading and elution buffers include arginine, the arginine concentration in the elution buffer is equal to or greater than about 15 mM, 20 mM, 25 mM, 30 mM, 35 mM, 40 mM, 45 mM, 50 mM, 75 mM, 100 mM, 150 mM/200 mM, 250 mM, 300 mM, 350 mM, 400 mM, 450 mM or 500 mM. In another embodiment where the loading, and elution buffers include arginine, the arginine concentration in the loading and elution buffers is between about 1 mM and about 100 mM. In another embodiment where the loading and elution buffers include arginine, the arginine concentration in the loading, and elution buffers is between about 50 mM and about 300 mM. In a related embodiment where the loading and elution buffers include arginine, the arginine concentration in the loading and elution buffers is between about 50 mM and about 200 mM.

In another embodiment, the amino acid is arginine, In one embodiment where the loading, wash and elution buffers include arginine, the arginine concentration is greater than about 1 mM, 2 mM, 3 mM, 4 mM, 5 mM, 6 mM, 7 mM, 8 mM, 9 mM or 10 mM. In another embodiment where the loading, wash and elution buffers include arginine, the arginine concentration in the elution buffer is equal to or greater than about 15 mM, 20 mM, 25 mM, 30 mM, 35 mM, 40 mM, 45 mM, 50 mM, 75 mM, 100 mM, 150 mM, 200 mM, 250 mM, 300 mM, 350 mM, 400 mM, 450 mM or 500 mM. In another embodiment where the loading, wash and elution buffers include arginine, the arginine concentration in the loading, wash and elution buffers is between about 1 mM and about 100 mM. In another embodiment where the loading, wash and elution buffers include arginine, the arginine concentration in the loading, wash and elution buffers is between about 50 mM and about 300 mM. In a related embodiment where the loading, wash and elution buffers include arginine, the arginine concentration in the loading and elution buffers is between about 50 mM and about 200 mM.

In one embodiment, the IEX chromatography or IEX column or IEX resin or IEX matrix is AEX chromatography or an AEX column or resin or matrix. In one embodiment, the AEX chromatography is carried out using (or the AEX matrix or material is) a matrix selected from the group consisting of Q Sepharose™ Fast Flow, DEAE Sepharose™ Fast Flow, ANX Sepharose™ 4 Fast Flow (high sub), Q Sepharose™ XL, Q sepharose big beads, DEAE Sephadex A-25, DEAE Sephadex A-50, QAE Sephadex A-25, QAE Sephadex A-50, Q sepharose high performance, Q sepharose XL, Sourse 15Q, Sourse 30Q, Resourse Q, Capto Q, Capto DEAE, Mono Q, Toyopearl Super Q, Toyopearl DEAE, Toyopearl QAE, Toyopearl Q, Toyopearl GigaCap Q, TSKgel SuperQ, TSKgel DEAE, Fractogel EMD TMAE, Fractogel EMD TMAE HiCap, Fractogel EMD DEAE, Fractogel EMD DMAE, Macroprep High Q, Macro-prep-DEAE, Unosphere Q, Nuvia Q, POROS HQ, POROS PI, DEAE Ceramic HyperD, and Q Ceramic HyperD.

In another embodiment, the IEX chromatography is CEX chromatography or a CEX column or resin or matrix. In one embodiment, the CEX chromatography is carried out using (or the CEX matrix or material is) a matrix selected from the group consisting of SP Sepharose™, CM Sepharose™, Toyopearl® SP 6.50M, and Fractogel® SO₃⁻. In a related embodiment, the CEX chromatography is carried but using (or the CEX matrix or material is) Fractogel SO3− SE HiCap (M), Fractogel COO− (M), YMC-BipPro S75, Capto S, SP Sepharose XL/FF, CM Sepahrose FF, SP/CM Toyopearl 650m, Toyopearl SP 550c, Toyopearl GigaCap, UNOsphere S, Eshmuno S, Macroprep High S, and POROS HS 50.

In one embodiment, prior to elution of the protein or polypeptide, the chromatography matrix, material or column or resin is subjected to a wash. In one embodiment, one or more of the loading, wash and elution buffer(s) have a pH of between about 4 and about 6.5. In a related embodiment, the pH of the loading, wash and/or elution buffers is between about 4.5 and about 6. In one embodiment, the pH of the loading buffer is between about between about 4 and about 6.5. In one embodiment, pH of the wash buffer is between about 4 and about 6.5. In one embodiment, pH of the elution buffer is between about 4 and about 6.5. In one embodiment, the loading, wash and/or elution buffer(s) is/are selected from the group consisting of an acetate buffer, a MES buffer, a citrate buffer and a bis tris buffer.

In one embodiment, prior to elution of the protein or polypeptide, the chromatography matrix, material or column or resin is subjected to a wash. In one embodiment, one or more of the loading, wash and elution buffer(s) have a pH of between about 6 and about 9. In a related embodiment, the pH of the loading, wash and/or elution buffers is between about 6.5 and about 8.5. In one embodiment, the pH of the loading buffer is between about between about 6 and about 9. In one embodiment, pH of the wash buffer is between about 6 and about 9. In one embodiment, pH of the elution buffer is between about 6 and about 9. In one embodiment, the loading, wash and/or elution buffer(s) is/are selected from the group consisting of a phosphate buffer, a MES buffer, a citrate buffer and a tris buffer.

In one embodiment, the IEX chromatography (or contacting or binding of the chromatography column or resin or matrix) is carried out at a temperature of between about 2° C. and about 30° C. In a related embodiment, the IEX chromatography (or contacting or binding of the chromatography column or resin or matrix) is carried out at a temperature of between about 2° C. and about 8° C. In a related embodiment, the IEX chromatography (or contacting or binding of the chromatography column or resin or matrix) is carried but at a temperature of between about 15° C. and about 25° C. In one embodiment, the column or resin residence time is between about 1 minute and about 24 hours. In another embodiment, the column or resin residence time is between about 1 minute and about 4 hours.

In one embodiment, the protein or polypeptide subjected to the isolation or purification methods exhibits “peak splitting” in chromatograms obtained using IEX (e.g., AEX or CEX) chromatography. In a related embodiment, the purified or isolated protein or polypeptide exhibits substantially reduced “peak splitting” after being subjected to the above purification or isolation methods.

In one embodiment, the protein or polypeptide is a recombinantly-produced protein or polypeptide. In one embodiment, the protein is a protein therapeutic molecule. In one embodiment, the therapeutic molecule is a peptide. Intone embodiment, the therapeutic molecule is a peptibody. In one embodiment, the therapeutic molecule is a domain-based protein. In one embodiment, the therapeutic molecule is an antibody or antigen-binding fragment thereof. In a related embodiment, the antibody is a monoclonal antibody (“mAb”) or antigen-binding fragment thereof. In a related embodiment, the monoclonal antibody is selected from the group consisting of an IgG1 mAb, an IgG2 mAb1nd an IgG4 mAb. In a related embodiment, the monoclonal antibody is a glycosylated antibody. In another embodiment, the monoclonal antibody is an aglycosylated antibody.

### BRIEF DESCRIPTION OF THE DRAWINGS

FIG. 1A is a chromatogram (A300 absorbance) of eluent from a cation exchange (“CEX”) Fractogel® SO₃⁻ chromatography column of mAb1 plotted as a function of elution sodium concentration showing absorbance at 300 nm with two distinct peaks (labeled “A” and “B”) and a graph of the percent of high molecular weight (“HMW”) species in the eluent.

FIG. 1B is a representation of the relative amounts of HMW species versus monomers in peaks A, B and the feed solution used to load the CEX column, as determined using analytical size exclusion chromatography

FIGS. 2A and 2B show data from analytical CEX HPLC experiments of mAb1 material eluted as either Peak A (FIG. 2A) or Peak B (FIG. 2B).

FIG. 3A shows data from re-chromatograph experiments of mAb1 peak A on Fractogel® SO₃⁻. The figure shows data from the original CEX run, as well as data from a re-chromatograph of peak A from the original run as well as data from a re-chromatograph of peak A from the first re-chromatograph run (as indicated).

FIG. 3B shows data from a re-chromatograph experiment of mAb1 peak B on Fractogel® SO₃⁻, including data from the original CEX run and data from a re-chromatograph of peak B from the original run (as indicated).

FIG. 3C shows the monomer and HMW concentration of material from the re-chromatographed Peak B shown in FIG. 3B.

FIG. 4 shows data from an evaluation of SP Sepharose™ (“SP FF”), CM Sepharose™ (“CM FF”), Toyopearl® SP 650M (“SP 650M”), and Fractogel® SO₃⁻ (“SO3−”) CEX of mAb1 with gradient elution.

FIG. 5 shows HMW mass balance and change in pH during elution relative to the load/wash pH plotted as a function of the buffer acetate concentration.

FIG. 6A shows the effects on % Peak B (from a CEX chromatogram of mAb1) of elution buffers differing in the type of anion (indicated).

FIG. 6B shows the elution profile with buffers having different anions (indicated) as a function of elution volume.

FIG. 7A shows the effects on % Peak B of load and elution flow rates (expressed as column residence times) from a CEX chromatography experiment on mAb1.

FIG. 7B shows the effects of mass loading (expressed as grams mAb1 per liter resin) on % Peak B and HMW mass balance.

FIG. 7C shows the effects of column residence time (expressed as column wash volumes-“CV”) on % Peak B of mAbs 3 and 17.

FIG. 8A shows mAb1 CEX chromatography elution profiles with load, wash, and elution buffers having different pH values as function of the elution salt concentration.

FIG. 8B shows the percent Peak B & HMW generation with load, wash, and elution buffers having different pH values (indicated).

FIG. 9A shows the results of mAb1 CEX chromatography runs at different temperatures (indicated).

FIG. 9B shows the percent HMW mass balance from the experiments shown in FIG. 9A as a function of the buffer/column temperature.

FIG. 10A shows a Fractogel® SO₃⁻ chromatogram of mAb1 in the presence of 500 mM glycine (labeled “500 mM glycine”) and a control Fractogel® SO₃⁻ chromatogram in the presence of acetate/NaCl (labeled “Control”). The addition of glycine to the CEX process reduced Peak B formation compared to the no glycine run.

FIG. 10B shows Fractogel® SO₃⁻ chromatograms of mAb1 in the presence of 5.0 mM arginine (labeled “50 mM arginine”), 100 mM arginine (labeled “100 mM arginine”), and Acetate/NaCl (labeled “Control”).

FIG. 11A shows the effects of various excipients (sucrose, proline, glycine and arginine) on % Peak B and HMW mass balance in the context of mAb1 CEX chromatography experiments.

FIG. 11B shows the effects on % Peak B and HMW mass balance of including arginine at the load/wash step, elution step, and both load/wash and elution steps (“Full process”).

FIGS. 12A and 12B are chromatograms showing the effect of including 125 mM arginine in bench scale runs of mAb1 purification. The funs were performed under identical conditions (acetate buffer at pH 5, mass load of 40 g mAb1 per mL resin, Fractogel SO3−, sodium chloride gradient elution) with the exception of the inclusion (FIG. 12B) or lack of inclusion (FIG. 12A) of 125 mM arginine in the load, wash and elution buffers.

FIG. 13 shows CEX chromatographic profiles with and without arginine. Each run was loaded to 20 g/L resin in 30 mM sodium acetate, pH 5.0 and eluted over a 20 CV linear gradient to 30 mM sodium acetate/1.0M sodium chloride, pH 5.0. Feed material was protein A pool that had been acid treated, neutralized and depth filtered. Arginine run was spiked with an arginine stock solution to 100 mM; the same volume of equilibration buffer was added to the no arginine control.

FIG. 14 shows starting % HMW and average Peak B area of experiments performed with 18 different mAbs. The x-axis indicates the mAb tested. The y-axis indicates the percent peak B during the elution during elution with 3 SD error bars from triplicate runs and the percent HMW in the starting sample. Those with elevated levels of peak B are circled. In this experiment, the mAbs were loaded on Fractogel SO3− at pH 5 in acetate buffer, washed, and then eluted with a sodium chloride gradient elution. The mAbs that showed a peak B percentage that was greater than the starting HMW were considered to have elevated levels of peak B.

### DETAILED DESCRIPTION

### Definitions

The terms “polypeptide” or “protein” are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have, been modified, e.g., by the addition of carbohydrate, residues to form glycoproteins, or phosphorylated. Polypeptides and proteins can be produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The terms “polypeptide” and “protein” specifically encompass peptibodies, domain-based proteins and antigen binding proteins, e.g., antibodies and fragments thereof, as well as sequences that have deletions from, additions to, and/or substitutions of one or more amino acids of any of the foregoing.

The term “polypeptide fragment” refers to a polypeptide that has an amino-terminal deletion, a carboxyl-terminal deletion, and/or an internal deletion as compared with the full-length protein. Such fragments may also contain modified amino acids as compared with the full-length protein. In certain embodiments, fragments are about five to 500 amino acids long. For example, fragments may be at least 5, 6, 8,10, 14, 20, 50, 70,100, 110, 150, 200, 250, 300, 350, 400, or 450 amino acids long. Useful polypeptide fragments include immunologically functional fragments of antibodies, including binding domains.

The term “antibody” refers to an intact immunoglobulin of any isotype, or an antigen binding fragment thereof that can compete with the intact antibody for specific binding to the target antigen, and includes, for instance, chimeric, humanized, fully human, and bispecific antibodies. An “antibody” as such is a species of an antigen binding protein. An intact antibody generally will comprise at least two full-length heavy chains and two full-length light chains, but in some instances may include fewer chains such as antibodies naturally occurring in camelids which may comprise only heavy chains. Antibodies may be derived solely from a single source, or may be “chimeric,” that is different portions of the antibody may be derived from two different antibodies. The antigen binding proteins, antibodies, or binding fragments may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and mutations thereof.

The term “antigen binding fragment” (or simply “fragment”) of an antibody or immunoglobulin chain (heavy or light chain), as used herein, comprises a portion (regardless of how that portion is obtained or synthesized) of an antibody that lacks at least some of the amino acids present in a full-length chain but which, is capable of specifically binding to an antigen. Such fragments are biologically active in that they bind specifically to the target, antigen and can compete with other antigen binding proteins, including intact antibodies, for specific binding to a given epitope. In one aspect, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of antigen binding proteins, including intact antibodies. Immunologically functional immunoglobulin fragments include, but are not limited to, Fab, Fab′, F(ab′)2, Fv, domain antibodies and single-chain antibodies, and may be derived from any mammalian source, including but not limited to human, mouse, rat, camelid or rabbit.

The term “cation exchange material” or “cation exchange matrix” or “cation exchange resin” refers to a solid phase that is negatively charged and has free cations for exchange, with cations in an aqueous solution passed over or through the solid phase. The charge may be provided by attaching one or more charged ligands to the solid phase, e.g. by covalent linking. Alternatively, or in addition, the charge may be an inherent property of the solid phase (e.g. as is the case for silica, which has an overall negative charge). Cation exchange material, matrix or resin may be placed or packed into a column useful for the purification of proteins.

The term “anion exchange material” or “anion exchange matrix” or “anion exchange resin” refers to a solid phase that is positively charged and has free anions for exchange with anions in an aqueous solution passed over or through the solid phase. The charge may be provided by attaching one or more charged ligands to the solid phase, e.g. by covalent linking. Alternatively, or in addition, the charge may be an inherent property of the solid phase. Anion exchange material, matrix or resin may be placed or packed into a column useful for the purification of proteins.

The term “buffer” or “buffered solution” refers to solutions which resist changes in pH by the action of its conjugate acid-base range. Examples of buffers that control pH at ranges of about pH 4 to about pH 6.5 include acetate, MES, citrate, bis tris, and other mineral acid or organic acid buffers; phosphate is another example of a buffer. Salt cations include sodium, ammonium, and potassium.

The term “loading buffer” or “equilibrium buffer” refers to the buffer containing the salt or salts which is mixed with the protein preparation for loading the protein preparation onto an IEX column. This buffer is also used to equilibrate the column before loading, and to wash to column after loading the protein.

The term “wash buffer” is used herein to refer to the buffer that is passed over the ion exchange material or matrix following loading of a composition or solution and prior to elution of the protein of interest. The wash buffer may serve to remove one or more contaminants from the ion exchange material, without substantial elution of the desired protein.

The term “elution buffer” refers to the buffer used to elute the desired protein from the column. As used herein, the term “solution” refers to either a buffered or a non-buffered solution, including water.

The term “washing” the ion exchange material or matrix means passing an appropriate buffer through or over the ion exchange material.

The term “eluting” a molecule (e.g. a desired protein or contaminant) from ah ion exchange material means removing the molecule from such material, typically by passing an elution buffer over the ion exchange material.

The term “contaminant” or “impurity” refers to any foreign or objectionable molecule, particularly a biological macromolecule such as a DNA, an RNA, or a protein, other than the protein being purified that is present in a sample of a protein being purified. Contaminants include, for example, other proteins from cells that secrete the protein being purified and proteins.

The term “separate” or “isolate” as used in connection with protein purification refers to the separation of a desired protein from a second protein or other contaminant or impurity in a mixture comprising both the desired protein and a second protein or other contaminant or impurity, such that at least the majority of the molecules of the desired protein are removed from that portion of the mixture that comprises at least the majority of the molecules of the second protein or other contaminant or impurity.

The term “purify” or “purifying” a desired protein from a composition or solution comprising the desired protein and one or more contaminants means increasing the degree of purity of the desired protein in the composition or solution by removing (completely or partially) at least one contaminant from the composition or solution.

The term “to bind” or “binding” a molecule to an ion exchange material means exposing the molecule to the ion exchange material or matrix under appropriate conditions (e.g., pH and selected salt/buffer composition) such that the molecule is reversibly immobilized in or on the ion exchange material or matrix by virtue of ionic interactions between the molecule and a charged group or charged groups of the ion exchange material or matrix.

The term “therapeutic biologic product” means a protein applicable to the prevention, treatment, or cure of a disease or condition of human beings. Examples of therapeutic biologic products include monoclonal antibodies, recombinant forms of a native protein such as a receptor, ligand, enzyme or cytokine, peptibodies, and/or a monomer domain binding proteins based on a domain selected from LDL receptor A-domain, thrombospondin domain, thyroglobulin domain, trefoil/PD domain, EGF domain, Anato domain, Notch/LNR domain, DSL domain, Anato domain, integrin beta domain, and Ca-EGF domain.

The term “peptibody” refers to a molecule comprising an antibody Fc domain (i.e., CH2 and CH3 antibody domains) that excludes antibody CH1, CL, VH, and VL domains as well as Fab and F(ab)₂, wherein the Fc domain is attached to one or more peptides, preferably a pharmacologically active peptide, particularly preferably a randomly generated pharmacologically active peptide. The production of peptibodies is generally described in PCT publication WO00/24782, published May 4, 2000.

HMW Species in IEX

During the development of a cation exchange step for the purification of an aglycosylated IgG1 (mAb1), unexpected elution profiles and significant HMW generation were observed. Aberrant peak shape and HMW generation have often been attributed to product denaturation upon binding to chromatographic media. Experiments performed in support of the present invention and methods described herein addresses, inter alia, development challenges arising from chromatography surface induced denaturation, the impact of typical IEX operational parameters on peak splitting and HMW generation, as well as mitigation strategies to allow the use of IEX without sacrificing product integrity or separation selectivity.

In particular, experiments performed in support of the present invention revealed “peak splitting”, with an unexpected second peak (termed “Peak B”) in certain chromatograms of monoclonal antibodies run on CEX columns. After testing a number of different hypotheses for the observation of peak B, it was determined Peak B was likely due to chromatographic surface induced protein denaturation, also termed “on-column” denaturation. This was a surprising finding, since IEX has been widely used with very few reported cases of suspected product denaturation.

Experiments performed in support of the present invention have further shown that operational pH, temperature, CEX resin, salt type, and column residence time all impact to some extent peak splitting and HMW generation of mAb 1. In contrast, buffer strength and mass loading did not have a significant impact on peak B formation. Surprisingly, it was found that the use of glycine or arginine, particularly arginine, in both the loading and elution phases of IEX chromatography, dramatically reduced peak B formation and HMW generation.

Chromatographic surface induced protein denaturation can be problematic in the purification and manufacture of certain biologic, therapeutics, e.g., monoclonal antibodies. For example, surface induced denaturation on chromatographic resins can create challenges in meeting typical quality attributes and may have implications for drug substance stability. Experiments performed in support of the present invention have led to the identification of excipients—e.g., glycine and arginine—which could be used to reduce or eliminate such denaturation. In particular, the addition of arginine at concentrations compatible with commercial protein therapeutic production processes was found to substantially eliminate chromatographic surface induced protein denaturation in CEX chromatography, as evidenced by, e.g., the substantial elimination of peak B formation. It was found that arginine limits the extent of denaturation and improves overall step yield without negatively impacting separation selectivity.

Exploring Possible Causes of Peak Splitting

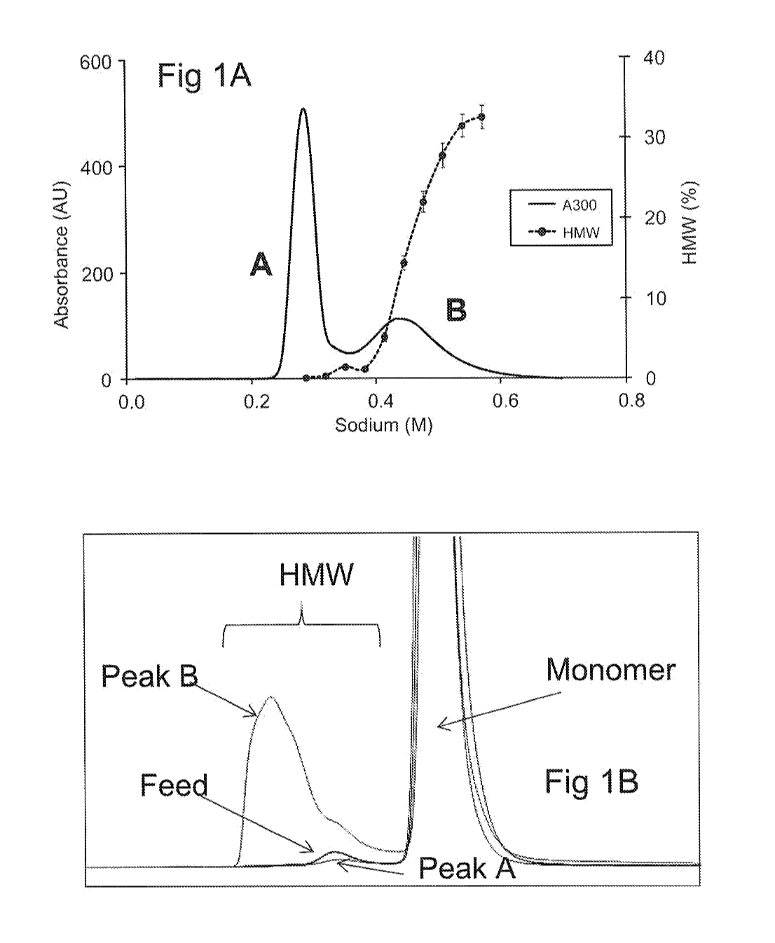

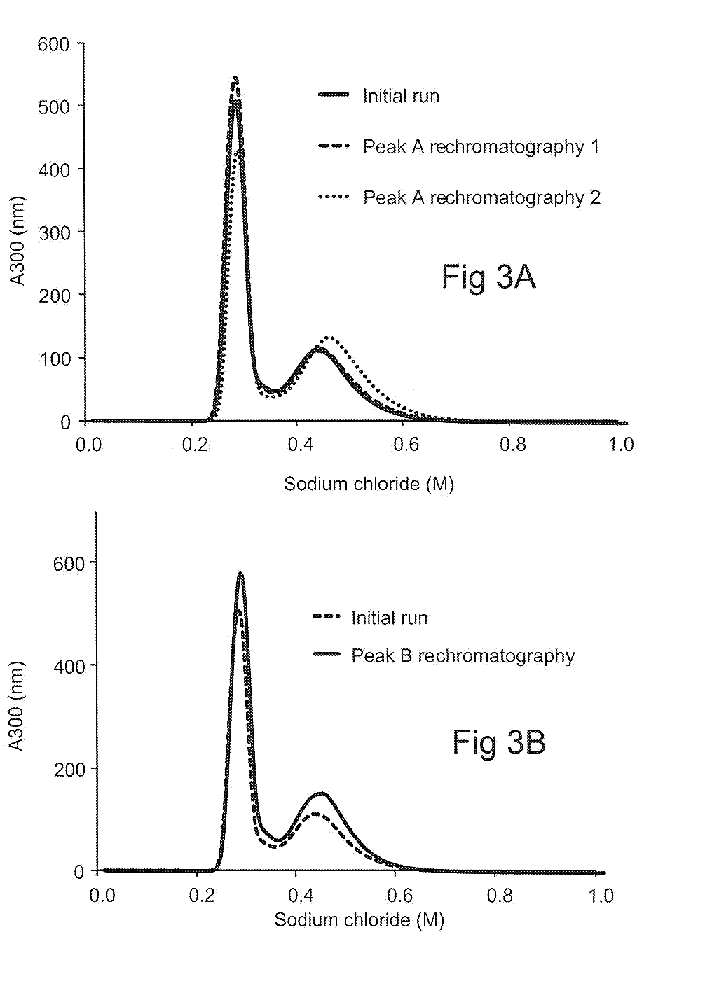

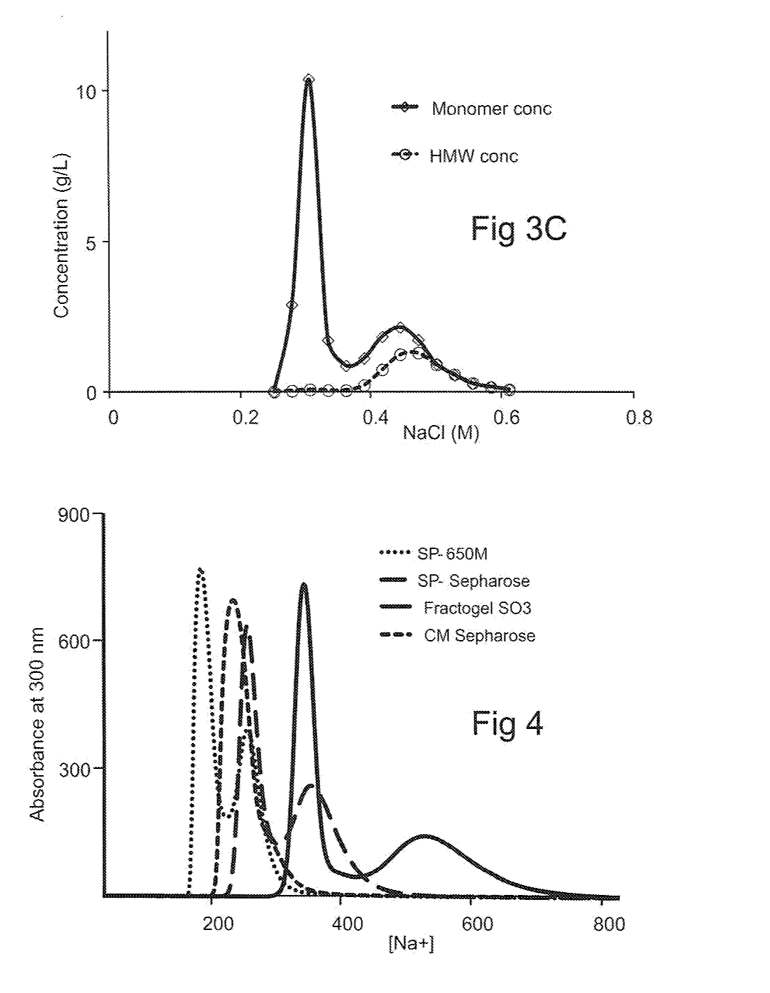

During development of mAb1 (an aglycosylated, IgG1), significant peak splitting was observed during cation exchange (CEX) chromatography (FIG. 1A). In addition to atypical peak shapes, the data showed significant aggregate formation (FIGS. 1A and 1B) that would be expected to manifest as yield loss in a manufacturing setting. Re-chromatography of the two peaks showed that peak splitting was occurring on the resin and was not separation of different species (FIGS. 3A, 3B and 3C). This was observed on several widely-used chromatographic media, including Fractogel® S0₃⁻, SP Sepharose™, and Toyopearl® SP 650M (FIG. 4).

Peak splitting and aggregate formation have significant implications for development of downstream processes. Aggregates are a major product related impurity that could be immunogenic and, therefore, are undesirable in therapeutic proteins and should be controlled during downstream process development. Generation of aggregates during common polishing chromatography steps may affect drug product quality (inability to remove aggregate), step yield (removal of aggregate), or product stability (molecule perturbations during chromatography may have adverse impact on long term stability).

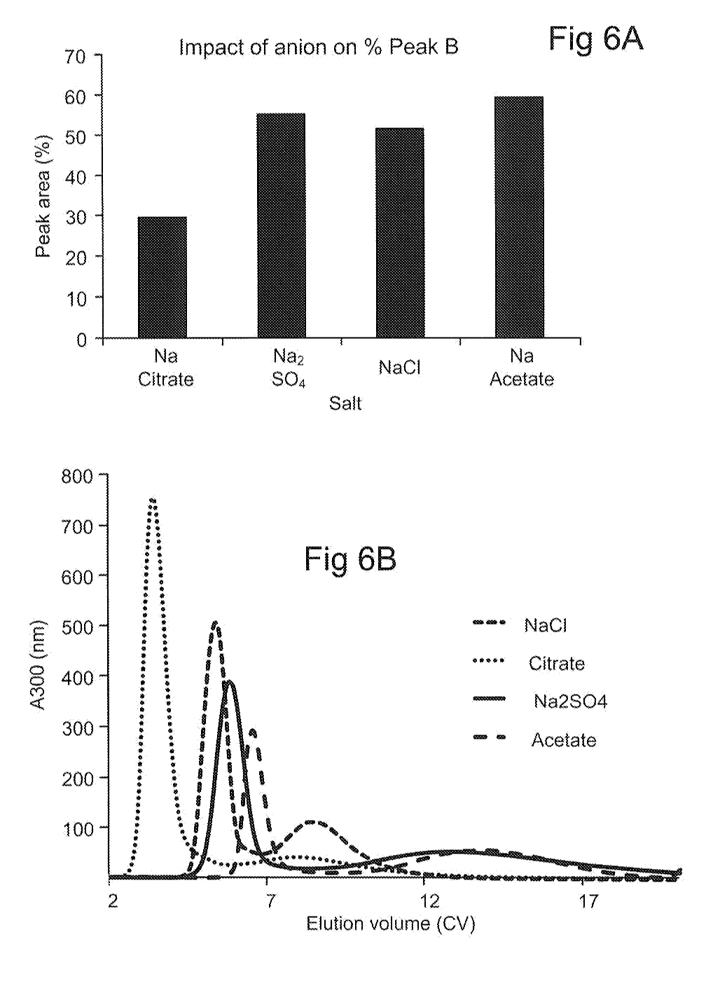

In order to address these issues, performing CEX chromatography in the presence of several salt systems and stabilizing excipients was evaluated. Varying the salt system did not have a significant impact on peak B formation (FIGS. 6A, 6B). Furthermore, other usual process development parameters surprisingly did not reduce peak B formation for a sufficiently robust process step.

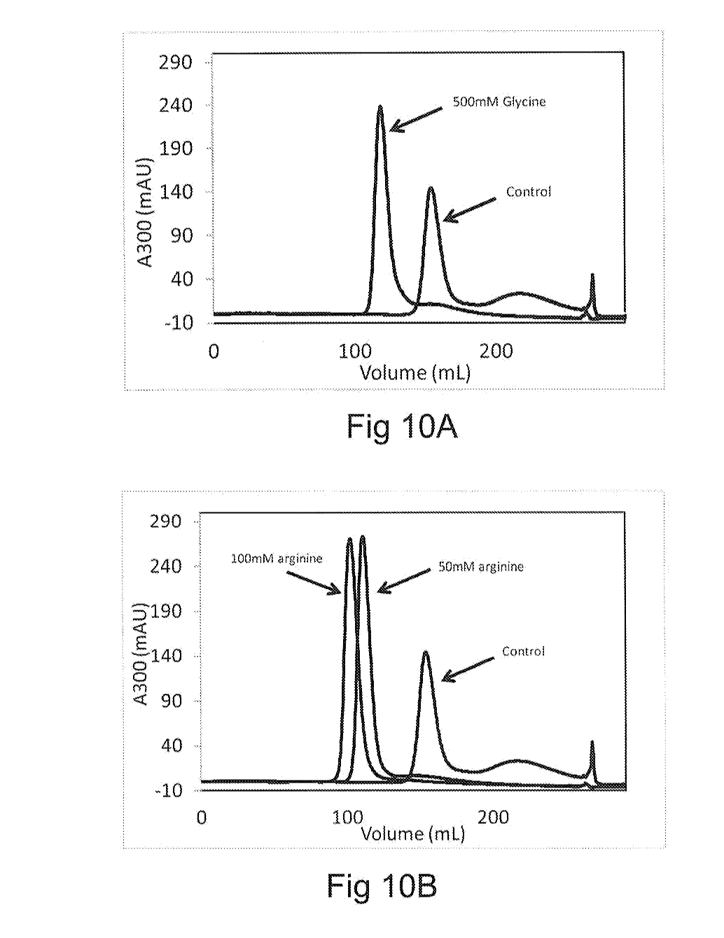

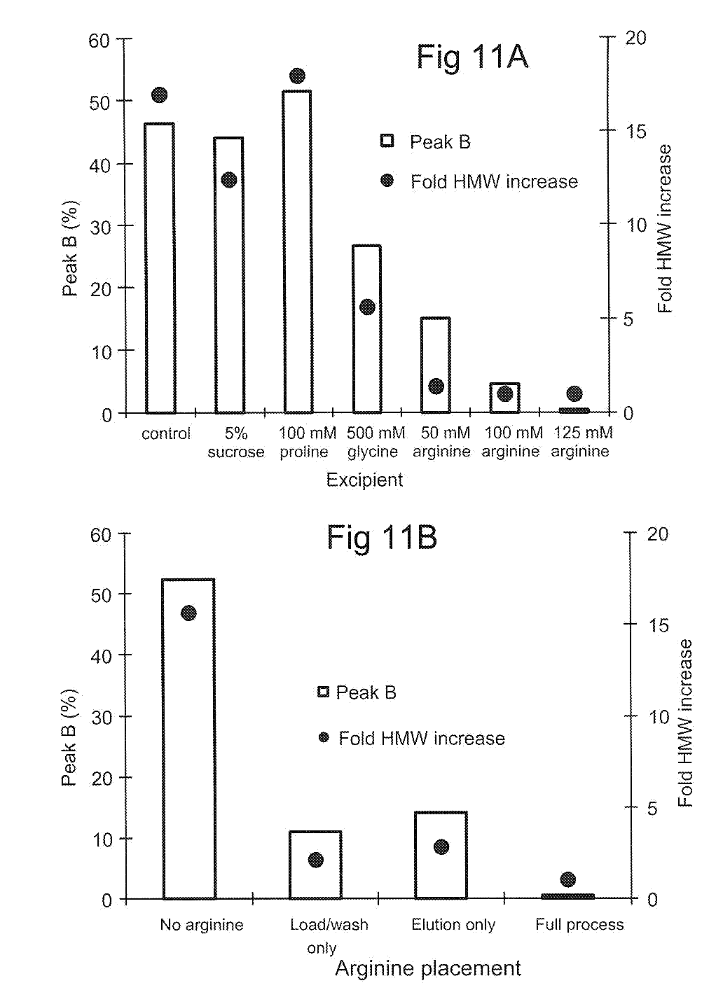

Experiments were then performed to evaluate stabilizing agents. As detailed below, sucrose and proline had no effect. Arginine and glycine, however, were found to reduce peak B formation and generation of HMW. In the case of mAb1, for example, a 50% reduction in peak B required 500 mM glycine (FIG. 10A). Experiments performed with other mAbs showed that in some cases, substantially lower concentrations of glycine, were effective at achieving Significant reductions in Peak B. Arginine at greater than about 100 mM dramatically reduced peak B formation and eliminated HMW generation/aggregate formation with mAb1 (FIGS. 10B, 11A).

Further, it was surprisingly found that arginine is needed in all phases of CEX operation (loading, wash and elution) to optimally control levels of Peak B & HMW generation (FIG. 11B). While not wishing to be bound any particular theory or mechanism, it appears that arginine inhibits HMW formation by decreasing binding site availability and inhibiting HMW formation upon elution.

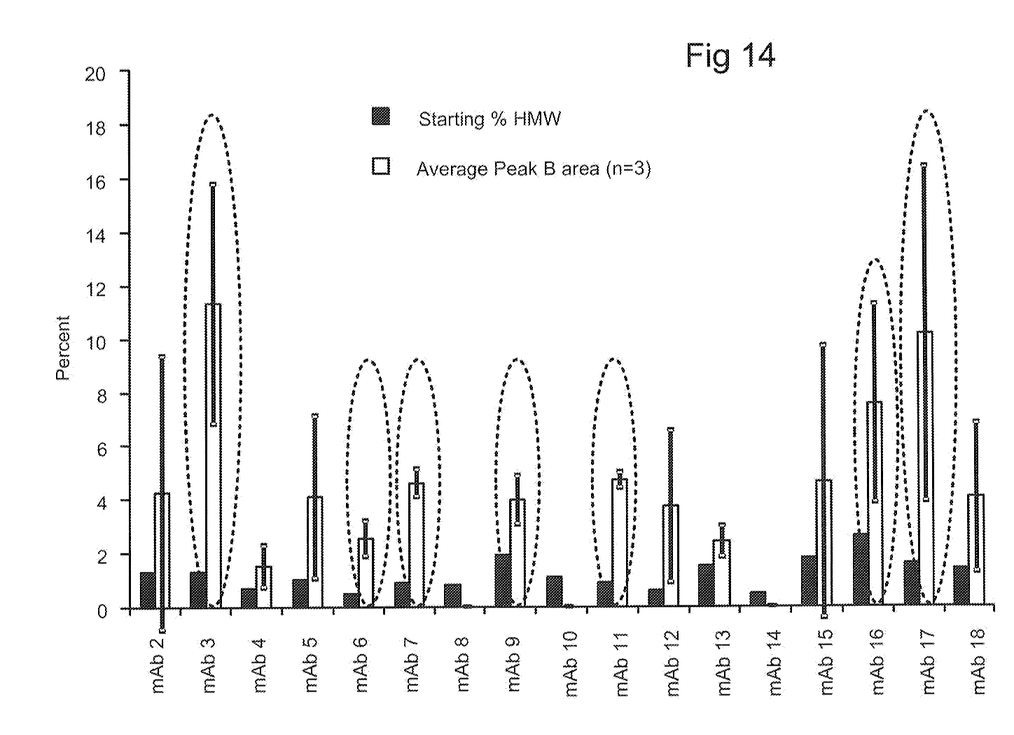

Subsequent experiments with other mAbs suggest that this phenomenon is not unique to mAb1 or aglycosylated molecules. Molecules that exhibit peak splitting on Fractogel® SO₃⁻ include a number of glycosylated IgG2 molecules as well (see, e.g., FIG. 14).

Ion Exchange Chromatography

The methods detailed herein are suitable for use in connection with ion exchange chromatography, including anion exchange (AEX) chromatography and cation exchange (CEX) chromatography. IEX is generally conducted using an ion exchange resin, which is typically packed into a column that may be used for protein purification according to standard methods.

Anion exchange (“AEX”) chromatography may be performed substantially as described in P. Gagnon, 1996, Purification tools for Monoclonal Antibodies, Validated Biosystems, Tucson, Ariz. Suitable resins, columns or matrixes that can be employed with AEX include, but are not limited to, Q Sepharose™ Fast Flow, DEAE Sepharose™ Fast Flow, ANX Sepharose™ 4 Fast Flow (high sub), Q Sepharose™ XL, Q sepharose big beads, DEAE Sephadex A-25, DEAE Sephadex A-50, QAE Sephadex A-25, QAE Sephadex A-50, Q sepharose high performance, Q sepharose XL, Sourse 15Q, Sourse 30Q, Resourse Q. Capto Q, Capto DEAE, Mono Q, Toyopearl Super Q, Toyopearl DEAE, Toyopearl QAE, Toyopearl Q, Toyopearl GigaCap Q, TSKgel SuperQ, TSKgel DEAE, Fractogel EMD TMAE, Fractogel EMD TMAE HiCap, Fractogel EMD DEAE, Fractogel EMD DMAE, Macroprep High Q, Macro-prep-DEAE, Unosphere Q, Nuvia Q, POROS HQ, POROS PI, DEAE Ceramic HyperD, and Q Ceramic HyperD.

Cation exchange (“CEX”) chromatography may be performed using standard methods substantially as described in P. Gagnon, (1996) supra, m& Yigzaw, Y, et al., (2009), Curr Pharm Biotechnol., 10 (4), 421-6). Suitable resins, columns or matrixes that can be employed with CEX include, but are not limited to, SP Sepharose™, CM Sepharose™. Toyopearl® SP 650M, and Fractogel® SO₃⁻. Additional suitable CEX resins, columns or matrixes include Fractogel SO3− SE HiCap (M), Fractogel COO− (M). YMC-BioPro S75, Capto S, SP Sepharose XL/FF, CM Sepahrose FF, SP/CM Toyopearl 650m, Toyopearl SP 550c, Toyopearl GigaCap, UNOsphere S, Eshmuno S, Macroprep High S, and POROS HS 50.

Optimizing Glycine, Arginine and/or Histidine Concentration

In any production process, the precise glycine, arginine and/or histidine concentration used may be optimized to balance inhibition of HMW generation with other performance parameters, such as, for example, impurity selectivity, dynamic binding capacity and viral clearance. In particular, it is expected that binding capacity may decrease using, e.g., glycine or arginine in the process—for example, in one set of experiments involving mAb1, it was observed that the addition of 100 mM arginine resulted in a binding capacity of 70 g/L resin, whereas the control column (with no added arginine) showed a binding capacity of 110 g/L resin. Using the guidance provided herein, one of skill in the art can readily perform optimization experiments to arrive at a desired balance of inhibition of HMW generation versus binding capacity. Similarly, viral clearance may be also impacted. For example, XMuLV is believed to bind to CEX resin; if arginine weakens the interactions with, the resin, it may also impact viral clearance. Further, conditions that decrease the protein retention may also impact where the virus elutes in relation to the product. As detailed herein, these parameters may be readily optimized by one of skill in the art with a few simple experiments, e.g., as exemplified below.

### Impurity Selectivity

During development, impurity selectivity may be assessed using many different methods. Relevant to these methods, an impure feed stock may be bound to the IEX resin and then eluted by either altering the pH, salt strength, pH and salt strength, or any other method that would disrupt the ionic interactions that lead to binding. This may be achieved by either step or gradient elution. In both cases, the fractions eluting off the column may be compared to the impurities in the feed material to assess removal of undesired material. Additionally, individual fractions across a single gradient elution may be analyzed to determine where the product of interest eluted compared to the impurities. These experiments may be carried out under a variety of conditions (pH, buffer type, salt type, mass load, residence time etc.) to determine the conditions that resulted in optimum resolution with acceptable step yield. Alternatively, selectivity may be evaluated under conditions in which the product of interest flows through the column during the binding phase while the impurities bind to the resin.

### Dynamic Binding Capacity

Dynamic binding capacity is generally determined by performing a frontal experiment at the target binding conditions. In these experiments, the product of interest may be loaded onto the equilibrated resin at a mass load (g product per L resin) which would be expected to, exceed the capacity. During loading, the column effluent is monitored to detect product break through. When break through is detected, the amount of protein that has bound to the resin is calculated and expressed as mass product bound per volume of resin.

### Viral Clearance

Viral clearance assessment of chromatography unit operations are typically performed on qualified scale down models of the chromatography step. During these studies, column operation is performed as is typical for the unit operation (buffer, pH, bed height, mass load, etc.). Prior to loading the feed material is spiked with a model virus (for example XMuLV is a common virus used to model endogenous retrovirus like particles (RVLP) expressed in mammalian cells). Then during the subsequent chromatography run, samples are taken and assayed for the presence of the virus. The amount of virus in the product containing pool is then compared to the amount loaded onto the column (and a hold control) to determine the amount of virus removed during the step. This is typically expressed as a log reduction value, or LRV.

### EXAMPLES

The following examples, including the experiments conducted and the results achieved, are provided for illustrative purposes only and are not to be construed as limiting the scope of the appended claims.

### Materials & Methods

Protein Preparation

The aglycosylated monoclonal IgG1 antibody mAb1 was expressed in CHO cells. The N-glycosylation site in the CH2 domain was removed by mutation of asparagine 297 to glutamine (N297Q). The experimental pI of mAb1 is 7.6 by cIEF. Unless otherwise noted, the mAb1 feed material was purified utilizing multiple chromatography steps to achieve a high purity stock solution (HMW<2%, HCP<50 ppm, DNA<LOD, <1% clipped species by rCE-SDS). Capture of mAb1 from harvested cell culture fluid (HCCF) was performed on MabSelect protein A resin (GE Healthcare, Piscataway, N.J., USA). The protein A elution pool underwent a low pH acid treatment step followed by neutralization to pH 5.0 and diatomaceous earth depth filtration to form the filtered viral inactivated pool (FVIP). The polishing steps were cation exchange chromatography using Fractogel SO3⁻ (EMD Biosciences, Gibbstown, N.J., USA) followed by hydrophobic interaction chromatography (HIC) using Phenyl Sepharose high sub (GE Healthcare, Piscataway, N.J., USA) operated in the flow through mode. The HIC pool was then concentrated to 70 g/L and buffer exchanged into a 9% sucrose solution buffered with 10 mM acetate at pH 5.2 by tangential flow filtration (TFF). For these studies, the purified protein stock solution was buffer exchanged into the desired CEX load conditions by TFF using a Millipore Pellicon 3 30 kD regenerated cellulose membrane (Billerica, Mass., USA).

Cation Exchange Chromatography

CEX chromatography was performed using standard methods substantially as described in P. Gagnon, (1996) supra, and Yigzaw, Y, et al., (2009), Curr Pharm Biotechnol., 10 (4), 421-6). CEX was generally carried out on material that had previously been passed over a Protein A column, subjected to a low pH viral inactivation step (60 min @ pH ˜3.6), and then brought back to neutral pH (“neutralized acid-treated pool”). In a typical experiment, 100 mL of a solution containing 50 mM sodium acetate, 1.0 M Arginine, pH 5.0 was added per liter of the neutralized acid-treated pool. The conditioned neutralized acid-treated pool was loaded onto a CEX column to a maximum of 30 g/L of resin. Product was typically eluted as a single fraction. Each CEX elution pool was filtered through a 0.2 μm filter and successively pooled into a holding tank.

The stationary phases Fractogel EMD SO3⁻ (M) and Fractogel EMD SO3⁻ (S) were, obtained from EMD Biosciences (Gibbstown, N.J., USA); Toyopearl SP-650M was obtained from Tosohaas (Montgomery, Pa., USA); SP Sepharose 4 fast flow and CM Sepharose were obtained from GE Healthcare (Piscataway, N.J., USA). Unless otherwise noted, all chromatography runs were performed using Fractogel SO3⁻ (M). Unless otherwise, specified, the conditions and parameters were as follows. Column diameter; as needed based on volume of material used. Bed height: 20+/−2 cm; Linear Flow Rate: 150 cm/hr for loading, 100 cm/hr for elution and strip; Loading: ≦30 mg/mL resin; UV monitor wavelength: 300 nm; Product Collection: start—OD=0.05, end—10% max OD.

The column was typically pre-equilibrated with 0.5M sodium acetate, pH 5.0, and then equilibrated with 75 mM sodium acetate, 0.1M arginine, pH 5.0. The load was typically a neutralized acid-treated pool as described above; Wash buffer: 75 mM sodium acetate, 0.1M arginine, pH 5.0; Elution buffer: 75 mM sodium acetate, 0.1M arginine, 0.125 M sodium sulfate, pH 5.0; Strip buffer: 0.2 M sodium hydroxide; Regeneration buffer: 0.5 M sodium hydroxide; and column storage buffer: 0.2 M sodium hydroxide.

All bench scale chromatography runs were conducted on an AKTA Explorer using Unicorn software version 5.01 (GE Healthcare, Piscataway, N.J., USA). CEX resins were packed into 1.1 cm ID Vantage columns (Millipore, Billirica, Mass., USA) to a bed height of approximately 20 cm and operated at a linear velocity of 140 cm/hr. The CEX columns were pre-equilibrated with 3 column volumes (CV) of 50 mM sodium acetate/1.0M sodium chloride, pH 5.0 followed by 3 CV of 50 mM sodium acetate, pH 5.0. The pH and conductivity of the column effluent was monitored to ensure that the resin was properly equilibrated. Highly purified mAb1 in 50 mM sodium acetate, pH 5.0 was loaded to 20 g/L resin. Following loading, the column was typically washed with 3 CV of 50 mM sodium acetate, pH 5.0. mAb1 was eluted over a 20 CV linear gradient from 50 mM sodium acetate, pH 5.0 to 50 mM sodium acetate/1.0M sodium chloride, pH 5.0. The elution peak was fractionated using a Frac-950 fraction collector. Absorbance of the protein was monitored at 280 and 300 nm. In-line pH and conductivity measurements were taken throughout the run. Any variation from the above method is noted in the text.

Analytical scale experiments were performed using the same operating conditions described above with a 0.4 cm ID by 10 cm height PEEK column (Applied Biosystems, Carlsbad, Calif., USA) and a Waters Alliance 2695 Separations Module equipped with a Waters 2996 Photodiode Array Detector (Milford, Mass.). Method control and integration were performed using Waters Empower 2 software (version 6.2).

All studies were performed at ambient temperature except where otherwise noted. Temperature controlled studies were performed in a walk-in temperature controlled room (Environmental Growth Chamber, Chagrin Falls, Ohio, USA). All solutions and columns were allowed to equilibrate to temperature set points prior to the chromatography runs.

HMW Determination

Calculation of % HMW

Levels of HMW in samples are determined by analytical size exclusion chromatography (see SEC analysis below). HMW is expressed as a percentage of the total protein content (e.g. % HWW+% monomer+% LMW=100%)

Calculation of HMW Mass Balance

HMW mass balance is determined by dividing the amount of HMW in the elution pool (and strip if applicable) by the amount of HMW loaded onto the column and expressed as a percentage. The amount of HMW in the load and elution is determined by multiplying the sample volume and product concentration and then multiplying by the fraction of the material that is measured as HMW by the SEC assay.

A280

The A280 method is used to determine protein concentration in purified samples. A product specific extinction coefficient is calculated based on the theoretical amino acid composition and is experimentally confirmed. Test samples are volumetrically diluted and the UV absorption at a wavelength of 280 nm is measured. Protein concentration is calculated using the Beer Lambert Law A=εbc (A=absorbance, ε=extinction coefficient, b=path length, c=concentration). Results are reported in mg/mL.

SEC Analysis

Size-exclusion HPLC separates proteins in solution based on their hydrodynamic volume with multimeric forms and aggregate peaks eluting earlier than the monomeric-form peak. Test samples and reference standard were injected onto a separation column at ambient temperature. Running buffer was 100 mM sodium phosphate/250 mM sodium chloride, pH 6.8. Flow rate was 0.5 mL/min. Samples were injected neat up to a 300 μg load. High molecular weight components were separated from the main component (monomer) using a Tosoh TSK-GEL G3000SWXL, 5 urn particle size, 7.8×300 mm size exclusion column. Components were eluted isocratically in a sodium phosphate and sodium chloride mobile phase. Eluted peaks were detected at 280 nm and integrated via HPLC software. The reference standard was analyzed as an assay control to identify any unexpected peaks and to assure the validity of the assay. Test sample results were reported as relative peak area percentages of high molecular weight component, main component (monomer), and low molecular weight component, if any. Fold HMW increase was calculated by summing the HMW mass across the entire elution peak and dividing by the starting HMW mass.

CEX HPLC Analysis

Ion-exchange HPLC separates variants based on differences in their surface charges. Under appropriate pH, charged proteins are separated on an ion-exchange column with a salt gradient elution. The eluent is, monitored by UV absorbance. Charge variants are separated by cation exchange chromatography (CEX) using a Dionex ProPac WCX-10 column. The protein is applied to the column in 20 mM sodium phosphate, pH 6.3 mobile phase with a flow rate of 0.8 mL/min. Charge variants are eluted using a linear gradient of 0-150 mM NaCl over 50 minutes with a total run time of 70 minutes. Eluted peaks are detected at 280 nm and integrated using chromatographic software.

### Example 1

### CEX Chromatography

Initial purification of mAb1 was performed using MabSelect protein A resin followed by low pH viral inactivation and depth filtration. The depth filtered viral inactivation pool (FVIP) had 3.9% HMW species and approximately 3000 ppm HCP. A sample (20 g mAb 1 per L resin) was subjected to CEX Fractogel® SO₃⁻ chromatography using an NaCl gradient from 0 mM NaCl to 500 mM NaCl buffered with 30 mM acetate, pH 5. Exemplary data are shown in FIG. 1A. One trace is absorbance at 300 nm, showing an atypical profile of two distinct peaks, labeled “A” and “B” on the plot. Also shown in FIG. 1A is a graph of the percent of high molecular weight (“HMW”) species in the eluent (error bars), showing that Peak B had a significantly larger percentage of high molecular weight (HMW) components, determined as described above.

Table 1, below, shows a summary of % yield, % HMW and HMW mass balance data from two experiments. Percent yield was calculated by dividing the total mass in the elution pool by the total mass loaded on the column (expressed as a percentage). % HMW and HMW mass balance were calculated as described above.

TABLE 1

| | | | HMW mass balance |
|---|---|---|---|
| Sample | Yield (%) | HMW (%) | (%) |
| Feed (1) | N/A | 1.2 | N/A |
| Feed (2) | N/A | 0.8 | N/A |
| Peak A (1) | 47 | 2.8 | 70% |
| Peak A (2) | 68 | 0.8 | 68% |
| Peak B (1) | 52 | 23 | 1600% |
| Peak B (2) | 31 | 21 | 830% |

The data show that % HMW and HMW mass balance were substantially greater in Peak B than in Peak A, indicating a larger fraction of HMW species in Peak B.

Analytical size exclusion chromatography (SEC) analysis was conducted on the feed and peak A & peak B material as described above. Exemplary data are shown in FIG. 1B. Note the significant increase in higher order High Molecular Weight (HMW) species in peak B as compared with peak A or feed.

Taken together, the data described above indicate that Peak B contains substantially more HMW species than Peak A.

### Example 2

### Peak A and B Characterization

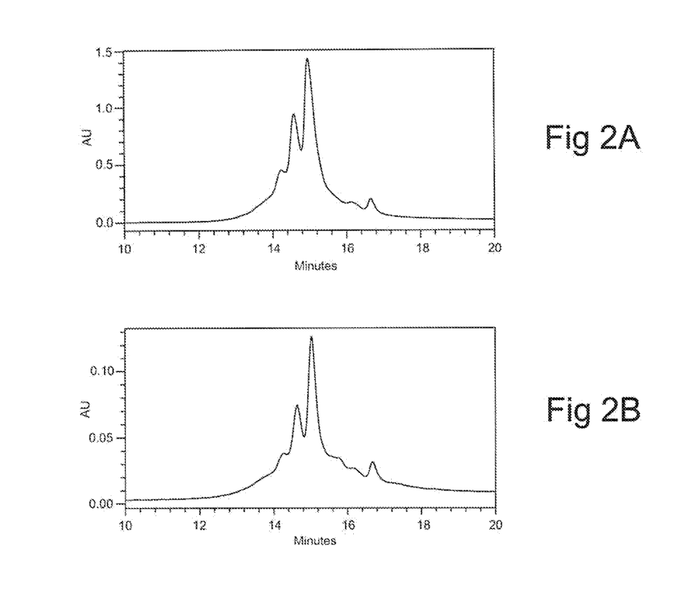

Results of analytical CEX HPLC experiments of material eluted as either Peak A or Peak B are shown in FIGS. 2A and 2B, respectively. The profiles of material from peak A (FIG. 2A) and peak B (FIG. 2B) are equivalent and indicate that the charge distribution composition of the material forming peak A and the material forming peak B is essentially identical. A Mass Spectrometry evaluation of the two peaks indicated that that the material, forming peak A and the material forming peak B has essentially the same mass. Taken together, these data strongly support the idea that the material forming peak A and the material forming peak B are essentially the same.

Peak A and B material was also evaluated for additional properties, including binding activity, peptide mapping and differential scanning calorimetry (“DSC”). These additional evaluations showed no differences (as between material from Peak A and material from Peak B) in binding activity, peptide mapping and DSC.

### Example 3

### Peak A and B Re-Chromatography

The two peaks were, collected and re-run on the same CEX column under the same operating conditions. Re-chromatography of peak A resulted in a similar profile as was seen with the original material, i.e., the formation of two distinct peaks (FIG. 3A). The first peak was re-chromatographed again, and again resulted in the formation of two prominent distinct peaks (FIG. 3A). Re-chromatography of peak B also resulted in a similar profile as was seen with the original material, i.e., the formation of two distinct peaks (FIG. 3B), and that a significant proportion of Peak B elutes as Peak A upon re-chromatography. Further, as can be appreciated from the data shown in FIG. 3C, the HMW distribution of the re-chromatographed Peak B is similar to that seen with the initial material.

Taken together, the data indicate that continued generation of peak B is not due to structural isoforms in the load, but is induced by the chromatography matrix surface in the column, i.e., is a consequence of on-column denaturation of the protein.

### Example 4

### Evaluation of Different Resin Backbones and Functional Groups

Different resins were evaluated for Peak B and HMW generation. FIG. 4 shows data from an evaluation of SP Sepharose™ (“SP FF”), CM Sepharose™ (“CM FF”), Toyopearl® SP 650M (“SP 650M”), and Fractogel® SO₃⁻ (“SO₃⁻”) with gradient elution from 0 mM NaCl to 600 mM NaCl buffered with 50 mM acetate, pH 5. All strong cation exchangers exhibit some level of peak splitting. The results are summarized in Table 3, below.

TABLE 3

| Resin | Peak B | HMW mass balance |
|---|---|---|
| TP SP-650M | 43% | 750% |
| CM Sepharose ™ | 15% | 320% |
| SP Sepharose ™ | 49% | 910% |
| Fractogel ® SO₃⁻ | 46% | 1700% |

As can be appreciated from the above data, CM Sepharose™, a weak cation exchanger, had the least % peak B and HMW generation.

### Example 5

### Buffer Strength does not Significantly Impact Peak B Formation

Transient pH shifts during step elutions have been shown to impact peak shape (Ghose, S., et al. pH Transitions in Ion-Exchange Systems: Role in the Development of a Cation-Exchange Process for a Recombinant Protein, Biotechnol. Prog. 18 (2002) 530-537)

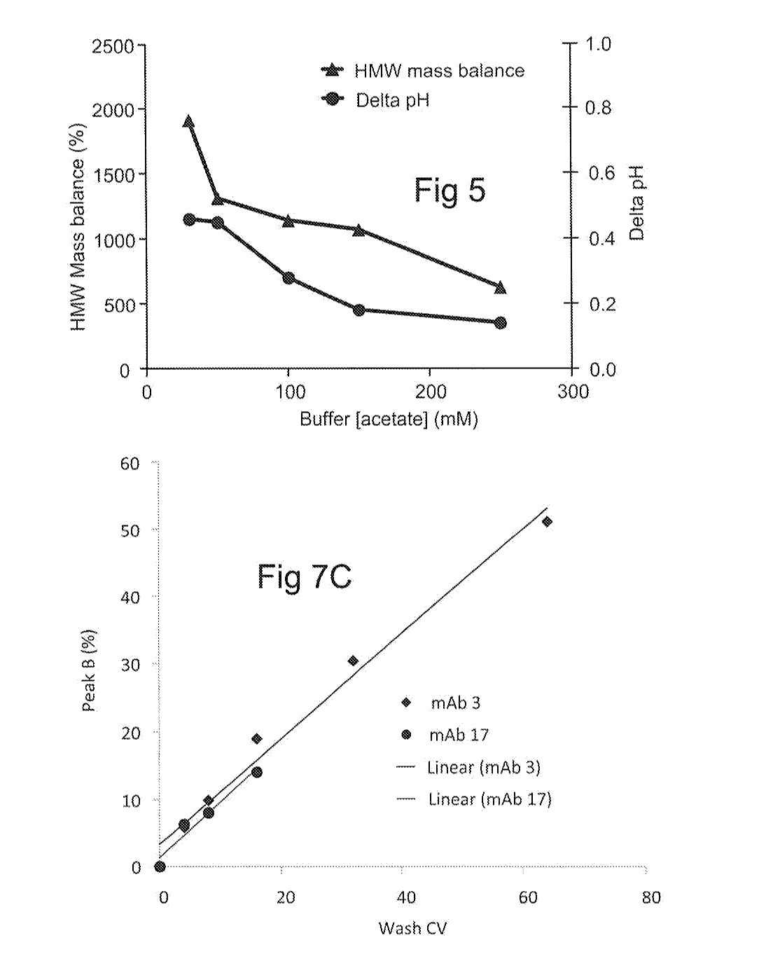

In this experiment, the buffer strength of the load, wash, and elution was examined from 50 mM through 250 mM sodium acetate, pH 5. The range of buffer strengths chosen was a practical range that would, be expected to mitigate pH transitions often seen during gradient elutions during CEX chromatography. Although there was a trend towards lower HMW generation with decreased pH transitions (FIG. 5), even when the transition was reduced from 0.5 units to ˜0.1 units, there was still 500% aggregate mass balance indicating that increasing the buffer concentration to the upper end of the practical limit would not be a practical option for minimizing peak B formation. This also demonstrates that peak B formation and HMW generation is not induced by transient pH swings during the elution phase.

As shown in FIG. 5, increasing buffer strength to minimize pH transitions did not dramatically improve HMW generation.

### Example 6

### Use of Different Eluting Salts Had Only Minor Impact on Peak B Formation

The type of anion in the elution buffer can impact chromatographic retention and % peak B on cation exchange systems. Experiments were performed as follows using the following buffers:

In each experiment, the mAb was loaded onto a column that was equilibrated in 50 mM acetate, pH 5. Following loading, the column was washed with equilibration buffer. Then the mAb was eluted over a linear gradient to 1.0 M sodium chloride, 1.0 M sodium citrate, 1.0M sodium sulfate, and 1.0M sodium acetate; all buffered with 50 mM acetate, pH 5.

FIG. 6A shows the Impact of anion on % Peak B. FIG. 6B shows the elution profile with different anions. As can be appreciated, citrate reduces the % of Peak B and thus helps improve step yield.

### Example 7

### Impact of Column Residence Time and Loading

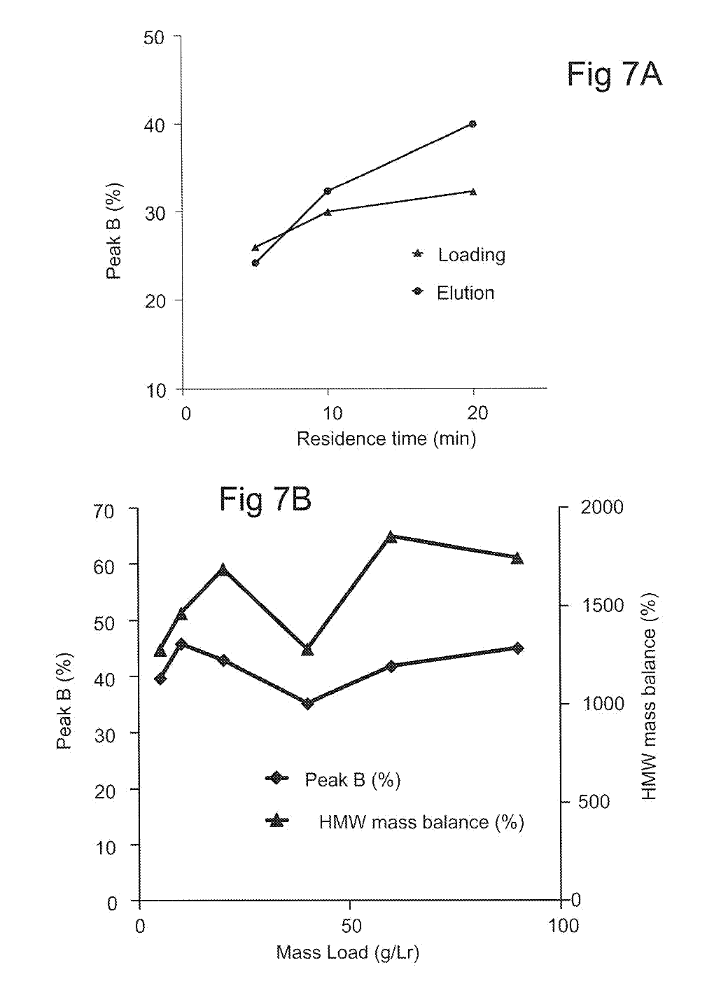

Columns were run with varying load and elution flow rates to determine the effect on % Peak B. During this study, the Fractogel SO3− was equilibrated with 50 mM acetate, pH 5 and then loaded to 40 grams mAb per liter of resin. Following loading the column was washed with 3 CV of equilibration buffer and then eluted over a linear gradient to 50 mM acetate/1.0M NaCl, pH 5. The residence time Was varied for either the load or the elution phase from 5 through 20 minutes. All other phases were performed at a 9 minute residence time. The percent peak B was determined by integration using chromatography software. The data shown in FIG. 7A, show that increasing column residence time increases % Peak B, and that the residence time during elution appears to have a greater impact than that during loading.

The effect of mass loading on % Peak B and HMW of mAb1 was evaluated. For each run, Fractogel SO3− was equilibrated (EQ) with 50 mM acetate, pH 5. Following EQ, the mAb was loaded to either to 5, 10, 20, 40, 60, or 90 grams mAb per liter resin and then washed with 3 column volumes of equilibration buffer. Following the wash phase, the mAb was eluted over a linear gradient to 50 mM acetate/1.0 M NaCl, pH 5. The percent peak B was determinedly integration using chromatography software, and the data are shown in FIG. 7B. Within the range examined (5-90 g/L resin), there was not a significant impact of mass leading on peak B % or HMW mass balance.

The impact of time bound to the column was also assessed. During these studies Fractogel SO3− was equilibrated (EQ) with 50 mM acetate, pH 5. Following EQ, the mAb was loaded onto the resin and then washed with 0, 4, 8, 16, 32, or 64 column volumes of equilibration buffer. Following the wash phase, the mAb was eluted over a linear gradient to 50 mM acetate/1.0 M NaCl, pH 5. The percent peak B was determined by integration using chromatography software. FIG. 7C shows the impact of time bound to the resin (expressed as wash volumes) on peak B percentage of mAb3 and mAb 17. Note that residence time correlates linearly to an increase in peak B percentage of mAbs 3 and 17.

### Example 8

### Increasing Operational pH Decreases Peak B Formation and HMW Generation

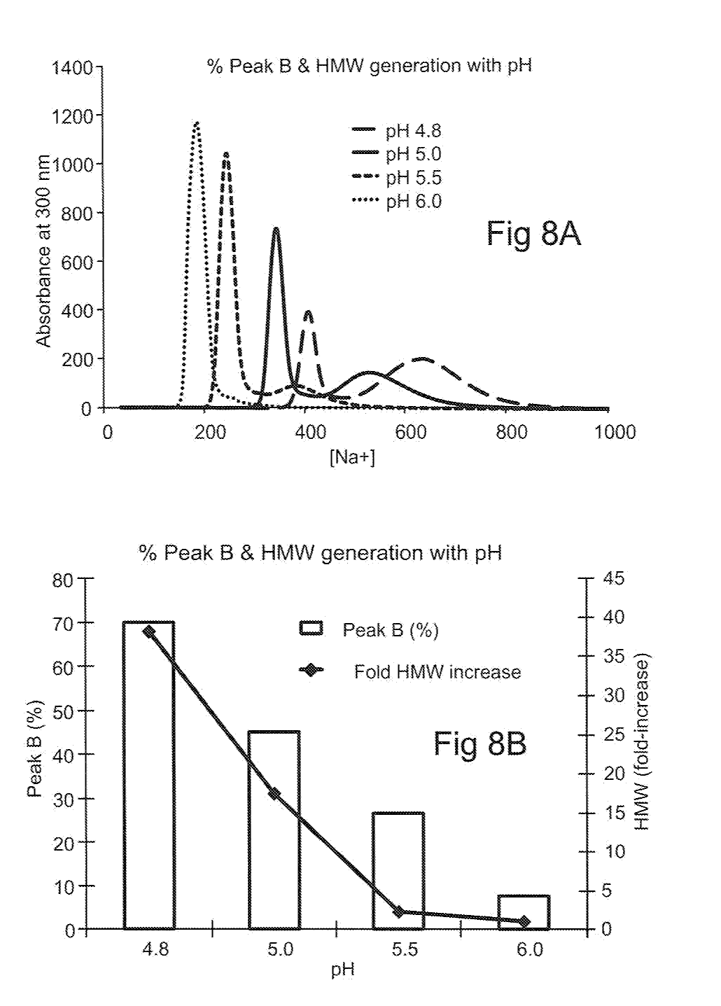

The effect of pH of the loading wash, and elution buffer on % Peak B and HMW was evaluated. In these experiments, the mAb was loaded onto a column that was equilibrated in either 50 mM acetate (pH 4.8, 5.0, or 5.5) or 50 mM MES (pH 6). Following loading, the column was washed with equilibration buffer. Then the mAb was eluted over a linear gradient to 1.0 M sodium chloride. The pH of the wash and elution phases was the same as the respective binding pH. The data are presented in FIGS. 8A and 8B. FIG. 8A shows the elution profiles as function of pH; FIG. 8B shows the percent Peak B & HMW generation with pH. As can be appreciated from the data, weaker binding and diminished capacity at higher pH reduced peak B formation (indicated as % Peak B) and HMW generation.

### Example 9

### Effects of Operational Temperature on Peak B and HMW Generation

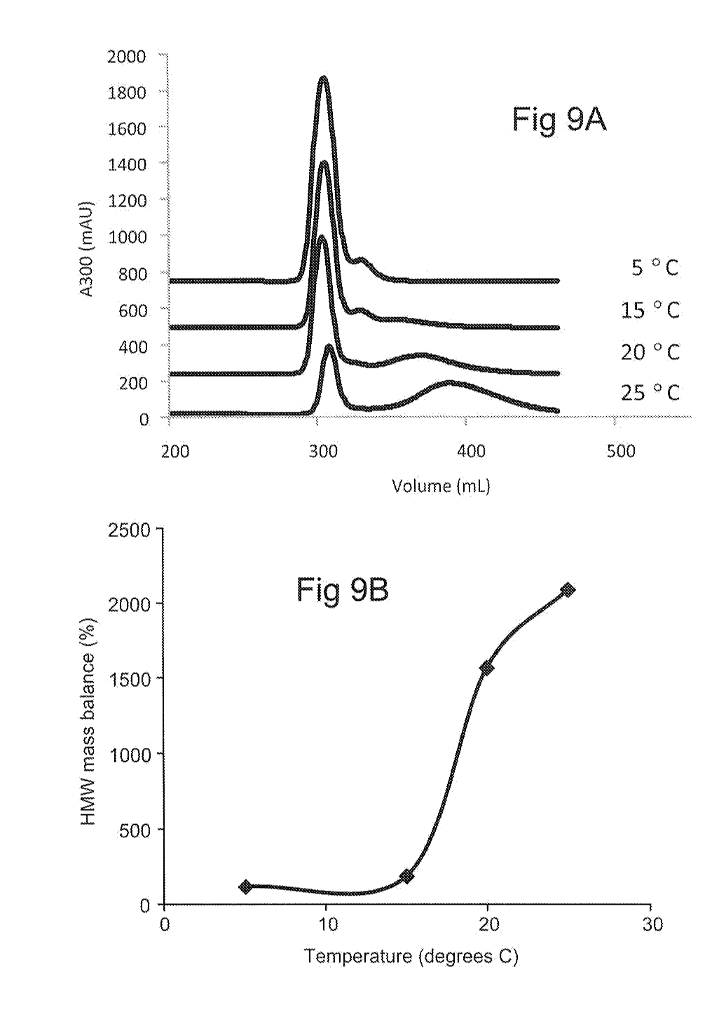

Columns were run under standard conditions (the mAb was loaded onto a column that was equilibrated in 50 mM acetate, pH 5. Following loading, the column was washed with equilibration buffer and then eluted over a linear gradient to 50 mM acetate/1.0M NaCl, pH 5.) at different temperatures (indicated) to determine the effect of column temperature on Peak B formation. The data are shown in FIGS. 9A and 9B. As can be appreciated from the data, % peak B and HMW generation decrease with decreasing temperature.

### Example 10

### Arginine and Glycine Inhibition of HMW Formation

A number of Stabilizing excipients, including sucrose, proline, arginine and glycine were tested for any effects on % peak B and HMW generation. The impact of adding sodium chloride and sodium sulfate to the feed was also examined and found to have no impact on peak splitting.

Exemplary data generated using mAb1 are shown in FIGS. 10A, 10B and 11A. Sucrose and proline had no effect (FIG. 11A), but as shown in FIGS. 10A, 10B and 11A, both arginine and glycine reduced peak B formation and HMW generation. Both glycine and arginine reduced % peak B. A 50% reduction in peak B was observed with the addition of about 500 mM glycine; other mAbs tested required a substantially lower glycine, concentration for a corresponding reduction in peak B. Arginine at greater than about 100 mM dramatically reduced peak B formation and eliminated HMW generation with mAb1 (FIG. 10A). In particular, no significant HMW generation was noted with arginine concentrations ≧100 mM.

Further, it was surprisingly found that arginine was needed in all phases of CEX operation (loading, wash and elution) to optimally control levels of Peak B & HMW generation. As shown in FIG. 11B, using arginine in either load only or elution only reduced % peak B of mAb1 to between about 10% and 15%, while including arginine in both steps reduced % peak B to less than 0.5%

### Example 11

### Optimized CEX Step Meets PQ Targets

Fractogel SO3− was equilibrated (EQ) with 75 mM acetate/100 mM arginine at pH 5. Following EQ, the feed material was conditioned to 100 mM arginine, pH 5 by the addition of a high concentration arginine stock solution and then loaded to 40 g mAb per L resin. Following loading, the column was washed with equilibration buffer. At the completion of the wash step the mAb was eluted with 75 mM acetate/125 mM sodium sulfate/100 mM arginine, pH 5

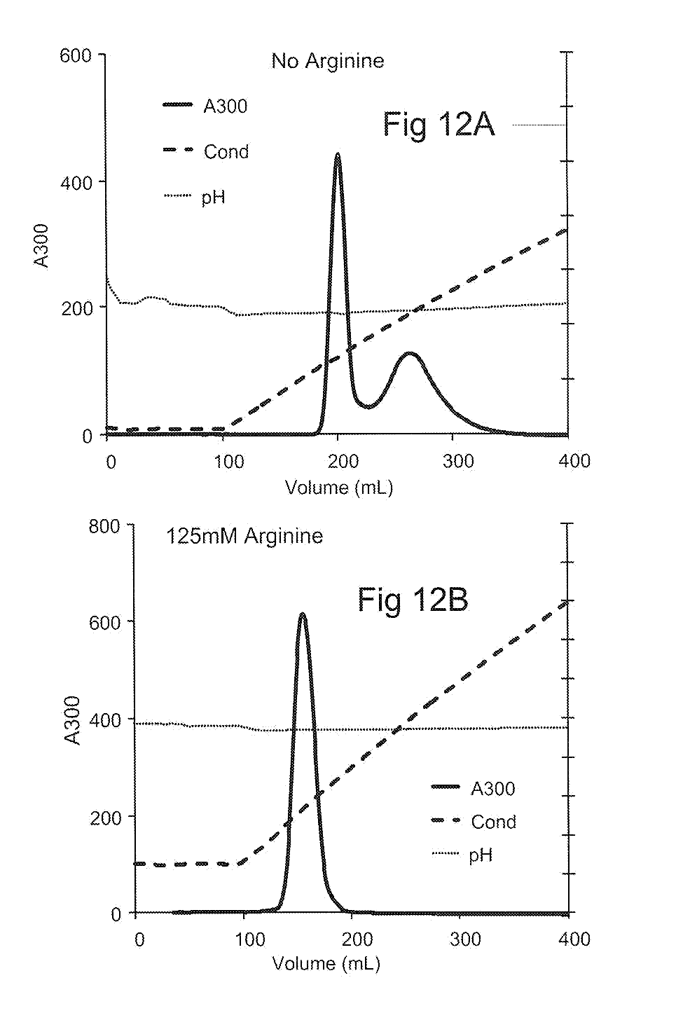

Exemplary data are shown in FIGS. 12A (no arginine) and 12B (125 mM arginine); the impact of including 100 mM arginine on certain quality attributes is summarized in Table 4, below. CEX chromatography in the presence of arginine met typical quality targets, including, viral clearance across the step.

TABLE 4

| Quality | | |
|---|---|---|
| attribute | Feed | Elution pool |
| Mass load | 40 g/L | NA |
| Yield | N/A | 90% |
| HCP | ~3000 ppm | 110 ppm |
| HMW | 2.0% | <1% |
| XMuLV LRV | NA | 4.4 logs |

### Example 12

### Application to the Purification Process

Experiments were performed to assess if a suitable degree of impurity removal was achievable in the presence of arginine when using a relevant feed stream. A representative feed stream for the CEX unit operation is depth filtered viral inactivation pool (FVIP). A CEX chromatography run was therefore performed in the presence of 100 mM arginine using FVIP as the feed and compared to the same process, without arginine to ascertain if acceptable product quality could be achieved on the CEX unit operation.

Fractogel SO3− was equilibrated (EQ) with 30 mM acetate/100 mM arginine at pH 5. Following EQ, the feed material was conditioned to 100 mM arginine, pH 5 by the addition of a high concentration arginine stock solution and then loaded to 20 g mAb per L resin. Each run was loaded to 20 g/L resin in 30 mM sodium acetate, pH 5.0 and eluted over a 20 CV linear gradient to 30 mM sodium acetate/1.0M sodium chloride, pH 5.0. Feed material was protein A pool that had been acid treated, neutralized and depth filtered. Arginine run was spiked with an arginine stock solution to 100 mM; the same volume of equilibration buffer was added to the no arginine control. Following loading, the column was, washed with equilibration buffer. At the completion of the wash step the mAb over a 20 column volume linear gradient to 30 mM acetate/100 mM arginine/1.0M sodium chloride, pH 5.

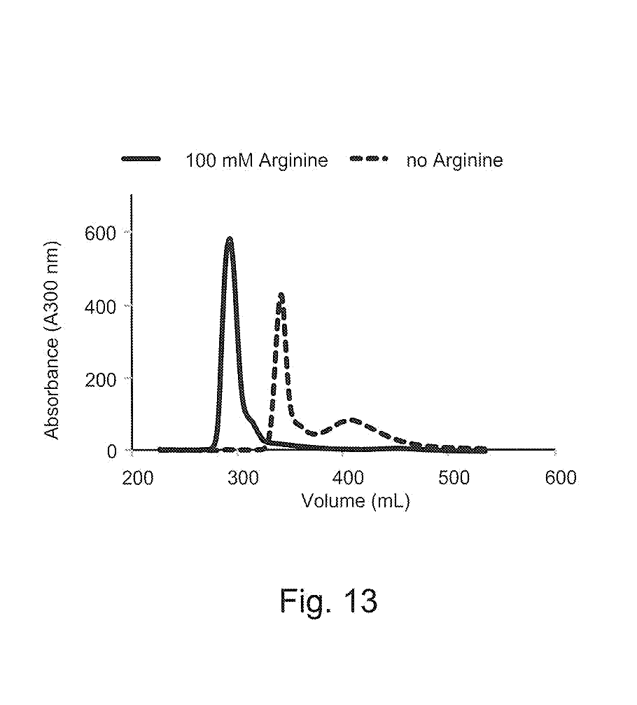

Chromatograms for these runs are shown in FIG. 13. Compared to the control (no arginine) run, it is clear that peak splitting is well controlled by the addition of 100 mM arginine in the process. The mAb eluted earlier in the gradient when run in the presence of 100 mM arginine.

The impact of including 100 mM arginine on certain quality attributes is summarized in Table 5, below. CEX chromatography in the presence of arginine controls denaturation on the column and also maintains acceptable selectivity for process and product related contaminants.

TABLE 5

| | | | Volume | HMW | |
|---|---|---|---|---|---|
| Run | Sample | Yield (%) | (CV) | (%) | HCP (ppm) |
| | Feed | NA | NA | 3.9 | 2778 |
| No arginine | Peak A | 46.5 | 2.0 | 1.8 | 826 |
| | Peak B | 46.1 | 7.6 | 31.0 | 3953 |
| | Peak A + B | 91.6 | 9.6 | 13.6 | 2205 |
| Arginine | Main peak | 91.3 | 2.3 | 2.3 | 401 |

### Example 13

### Applicability to Different Antibodies

Experiments were done to assess the applicability of the CEX-Arg approach to different antibodies. Fractogel® SO₃⁻ was used for the evaluation. The data are shown in FIG. 14. Seven out of 18 mAbs tested (circled in the Figure) had elevated levels of peak B, indicating that the methods described above may be applicable to a wide range of proteins & polypeptides.

In this example, for each run Fractogel SO3− was equilibrated (EQ) with 30 mM acetate, pH 5. Following EQ, the mAb was loaded and then washed with 3 column volumes of equilibration buffer. Following the wash phase, the mAb was eluted over a linear gradient to 30 mM acetate/1.0M NaCl, pH 5. The percent peak B was determined by integration using chromatography software. Each mAb was run in triplicate. The percent peak B was compared to the starting HMW content of the sample. In the cases where the percent peak B (including 3 standard deviation error bars) exceeded the starting level of HMW are indicated.

1. A method of reducing high molecular weight species (HMW) formation in a sample containing a protein purified using ion exchange (IEX) chromatography, comprising
loading the protein, in a loading buffer containing at least 1 mM of one or more amino acids selected from the group consisting of arginine and glycine, onto an IEX resin, and
eluting the protein off the IEX resin using an elution buffer containing at least 1 mM of one or more amino acids selected from the group consisting of arginine and glycine,
wherein presence of said one or more amino acids in said loading and elution buffers reduces HMW formation in said sample as compared with a sample of a protein purified using IEX chromatography with loading and elution buffers that do not contain al least 1 mM of one or more amino acids selected from the group consisting of arginine and glycine.

2. The method of claim 1, wherein the IEX resin is in an IEX column.

3. The method of claim 1, further comprising washing said column or resin with a wash buffer between said loading and said eluting, wherein said wash buffer contains at least 1 mM of one or more amino acids selected from the group consisting of arginine and glycine.

4. The method of claim 1, wherein each of said buffers contain at least 10 mM of one or more amino acids selected from the group consisting of arginine and glycine.

5. The method of claim 4, wherein said one or more amino acids is glycine and each of said buffers contain at least 100 mM glycine.

6. The method of claim 4, wherein said one or more amino acids is arginine.

7. The method of claim 6, wherein each of said buffers contain at least 20 mM arginine.

8. The method of claim 1, wherein said IEX column or resin is an anion exchange (AEX) column or resin.

9. The method of claim 8, wherein said AEX column or resin is selected from the group consisting of Q Sepharose Fast Flow, DEAE Sepharose Fast Flow, ANX Sepharose 4 Fast Flow, Q Sepharose XL, Q Sepharose big beads, DEAE Sephadex A-25, DEAE Sephadex A-50, QAE Sephadex A-25, QAE Sephadex A-50, Q sepharose high performance, Q sepharose XL, Sourse 15Q, Sourse 30Q, Resourse Q, Capto Q, Capto DEAE, Mono Q, Toyopearl Super Q, Toyopearl DEAE, Toyopearl QAE, Toyopearl Q, Toyopearl GigaCap Q. TSKgel SuperQ, TSKgel DEAE, Fractogel EMD TMAE, Fractogel EMD TMAE HiCap, Fractogel EMD DEAE, Fractogel EMD DMAE, Macroprep High Q, Macro-prep-DEAE, Unosphere Q, Nuvia Q, POROS HQ, POROS PI, DEAE Ceramic HyperD, and Q Ceramic HyperD.

10. The method of claim 1, wherein said IEX column or resin is a cation exchange (CEX) column or resin.

11. The method of claim 10, wherein said CEX column or resin is selected from the group consisting of SP Sepharose, CM Sepharose, Toyopearl SP 650M, and Fractogel SO₃⁻.

12. The method of claim 10, wherein said CEX column or resin is selected from the group consisting of Fractogel SO3− SE HiCap (M), Fractogel COO− (M), YMC-BioPro S75, Capto S, SP Sepharose XL/FF, CM Sepharose FF, SP/CM Toyopearl 650m, Toyopearl SP 550c, Toyopearl GigaCap, UNOsphere S, Eshmuno S, Macroprep High S, and POROS HS 50.

13. The method of claim 1, wherein each of said buffers has a pH of between 4.0 and 6.5.

14. The method of claim 1, wherein each of said buffers is selected from the group consisting of an acetate buffer, a MES buffer, a citrate buffer and a bis tris buffer.

15. The method of claim 1, wherein the method is carried out at a temperature of between 2° C. and 8° C.

16. The method of claim 1, wherein the method is carried out at a temperature of between 15° C. and 25° C.

17. The method of claim 1, wherein the column residence time is between 1 minute and 4 hours.

18. The method of claim 1, wherein the protein is a recombinantly-produced protein or polypeptide.

19. The method of claim 1, wherein the protein is selected from the group consisting of a peptibody, a domain-based protein, and a monoclonal antibody or antigen-binding fragment thereof.

20. The method of claim 19, wherein the protein is a therapeutic monoclonal antibody (mAb) selected from the group consisting of an IgG1 mAb, an IgG2 mAb1nd an IgG4 mAb.

21. The method of claim 20, wherein said mAb is aglycosylated.

22. The method of claim 21, wherein said mAb is an aglycosylated IgG1 mAb.

23. The method of claim 1, used in a downstream process purification of a therapeutic biologic product.
